(19) United States
(12) Patent Application Publication
(10) Pub. No.: US 2026/0191706 A1
(43) Pub. Date: Jul. 9, 2026

(54) REFASTENABLE ABSORBENT ARTICLES WITH ABSORBENT CHASSIS AND BELT BONDING ARRANGEMENTS

(71) Applicant: The Procter & Gamble Company, Cincinnati, OH (US)

(72) Inventors: Koichi MORIMOTO, Cincinnati, OH (US); Bin WANG, Beijing (CN)

(21) Appl. No.: 19/551,781

(22) Filed: Feb. 27, 2026

Related U.S. Application Data
(63) Continuation of application No. PCT/CN2023/123216, filed on Oct. 7, 2023.
(60) Provisional application No. 63/540,448, filed on Sep. 26, 2023.

(51) Int. Cl. A61F 13/56; A61F 13/49; A61F 13/496

(52) U.S. Cl. CPC A61F 13/5638; A61F 13/49011; A61F 13/496; A61F 2013/49025

(57) ABSTRACT

Absorbent articles comprising refastenably connected first and second belts. Longitudinally opposing end regions of the chassis are connected with the first and second belts, and a portion of the chassis that overlaps the first belt defines a chassis overlap region. The chassis overlap region comprises an attachment area where adhesive is positioned between the chassis and the wearer facing surface of the first belt. The attachment area comprises a first attachment zone adjacent to an inner laterally extending edge of the first belt and a second attachment zone longitudinally outboard of the first attachment zone. The first attachment zone has a first lateral width and the second attachment zone has a second lateral width. The first lateral width is less than the second lateral width. The first belt also comprises a first lower elastic zone having a tensile force of from 0.10 N to 1.20 N.

### CROSS REFERENCE TO RELATED APPLICATION

This application is a continuation of Patent Application No. PCT/CN2023/123216, filed on Oct. 7, 2023, which claims the benefit of U.S. Provisional Application No. 63/540,448, filed on Sep. 26, 2023, which are incorporated herein by reference.

### FIELD

The present disclosure relates to absorbent articles, and more particularly, to refastenable absorbent articles with absorbent chassis and belt bonding arrangements in the front waist region including one or more non-bonded regions.

### BACKGROUND

Infants and other incontinent individuals wear absorbent articles to receive and contain urine and other body fluids such as feces. Some caregivers may prefer refastenable pant-style disposable articles to enable convenient and hygienic removal and disposal of the soiled absorbent articles. Current disposable refastenable pant-style absorbent articles generally include an absorbent chassis connected with front and back elastic belts, wherein opposing end regions of the front and back belts are refastenably connected with each other at side seams. When worn, the absorbent article is configured such that the front and back belts encircle the waist of the wearer and the chassis extends from the front of the body to the back with a crotch portion positioned between the wearer's legs. While the curvature of the chassis around the body may provide a close fit which effectively contains body fluids, it may cause an undesirable tension on the front belt. In some instances, the bottom of the front belt can be pulled towards the wearer's thigh, resulting in an inward curling of a portion of the fastener component. This can cause the hooks of the fastener component to contact the body and may irritate the skin causing the wearer to experience discomfort and/or undesirable skin markings.

There is a need for disposable refastenable pant-style absorbent articles that provide a comfortable and close fit yet avoids contact between the skin and the hooks of the fastener component during use.

### SUMMARY

The present disclosure solves the problem of the undesirable and uncomfortable fastener component inward curl and hook-to-skin contact caused during wear of refastenable pant-style articles by providing chassis and belt bonding arrangements that disconnect a portion of the first belt from the chassis at one or more non-bonded regions.

Described herein is an absorbent article comprising: a chassis comprising a topsheet, a backsheet, and an absorbent core positioned between the topsheet and the backsheet. The chassis further comprising a first end region and a second end region longitudinally separated from the first end region by a crotch region. The absorbent article further comprising a first belt comprising a first end region and a second end region laterally separated from the first end region by a central region, and a second belt comprising a first end region and a second end region laterally separated from the first end region by a central region. The first end region of the chassis is connected with the central region of the first belt, and the second end region of the chassis is connected with the central region of the second belt. The first end region and the second end region of the first belt each comprise a fastener component. The fastener components are refastenably connected with the first and second end regions of the second belt to form a waist opening and two leg openings. The first and second belts each comprise: a wearer facing surface and a garment facing surface; an outer laterally extending edge extending along a portion of the waist opening; an inner laterally extending edge extending along a portion of each of the leg openings; and a first substrate, a second substrate, and a plurality of elastic strands positioned between and connected with the first substrate and the second substrate. A portion of the chassis overlaps the wearer facing surface of the first belt to define a chassis overlap region. The chassis overlap region comprises an attachment area where adhesive is positioned between the chassis and the wearer facing surface of the first belt and bonds the chassis with the first belt. The attachment area comprises a first attachment zone adjacent to the inner laterally extending edge of the first belt and a second attachment zone positioned longitudinally outboard of the first attachment zone. The first attachment zone comprises a first lateral width and the second attachment zone comprises a second lateral width. The first lateral width is less than the second lateral width. The first belt comprises a first lower elastic zone extending in a lateral direction and positioned longitudinally inboard of the second attachment zone and laterally outboard of the first attachment zone and a first upper elastic zone extending in the lateral direction and positioned longitudinally outboard of the first lower elastic zone. The first lower elastic zone has a tensile force of 0.10 N to 1.20 N.

### BRIEF DESCRIPTION OF THE DRAWINGS

FIG. 1 shows a perspective view of a refastenable diaper pant in a pre-fastened configuration.

FIG. 2A shows a top view of the diaper pant of FIG. 1.

FIG. 2B shows a top view of the diaper pant of FIG. 2A in a compressed state.

FIG. 2C1 is a cross-sectional view of the diaper pant of FIG. 2B taken along line 2C-2C showing details of a flange connection with a first configuration of belt laminate structures.

FIG. 2C2 is a cross-sectional view of the diaper pant of FIG. 2B taken along line 2C-2C showing details of a flange connection with a second configuration of belt laminate structures.

FIG. 2C3 is a cross-sectional view of the diaper pant of FIG. 2B taken along line 2C-2C showing details of a flange connection with a third configuration of belt laminate structures.

FIG. 2C4 is a cross-sectional view of the diaper pant of FIG. 2B taken along line 2C-2C showing details of a flange connection with a fourth configuration of belt laminate structures with the second belt provided with a stiffening patch.

FIG. 2C5 is a cross-sectional view of the diaper pant of FIG. 2B taken along line 2C-2C showing details of a flange connection with a fifth configuration of belt laminate structures with the second belt provided with a stiffening patch.

FIG. 2C6 is a cross-sectional view of the diaper pant of FIG. 2B taken along line 2C-2C showing details of a flange connection with a sixth configuration of belt laminate structures with the second belt provided with a stiffening patch.

FIG. 2D is a detailed sectional view of a bond structure between substrates of the flange and first belt comprising substantially tackifier free adhesive.

FIG. 2E is a detailed view of the bond structure of FIG. 2D applied between two nonwoven substrates.

FIG. 3A shows a plan view of a diaper pant with the portion of the diaper that faces away from a wearer oriented toward the viewer.

FIG. 3B shows a plan view of a diaper pant with the portion of the diaper that faces toward a wearer oriented toward the viewer.

FIG. 3C shows a plan view of a diaper pant with the portion of the diaper that faces away from a wearer oriented toward the viewer, illustrating example elastic material arrangements in the first and second belts.

FIG. 3D shows a plan view of a diaper pant with the portion of the diaper that faces away from a wearer oriented toward the viewer, illustrating first and second belt size and shape features.

FIG. 3E shows a plan view of a diaper pant with the portion of the diaper that faces away from a wearer oriented toward the viewer, illustrating first and second belt size and shape features.

FIG. 3F shows a plan view of a diaper pant with the portion of the diaper that faces away from a wearer oriented toward the viewer, illustrating first and second belt size and shape features.

FIG. 4 is a cross-sectional view of the diaper pant of FIG. 3A taken along line 4-4 showing first and second elastic belts provided with panel layers.

FIG. 4A is a cross-sectional detailed view of a first belt provided with panel layers wherein one panel layer is folded over another panel layer.

FIG. 4A1 is a cross-sectional detailed view of another example configuration wherein the first belt is provided with panel layers wherein one panel layer is folded over another panel layer.

FIG. 4A2 is a cross-sectional detailed view of another example configuration wherein the first belt is provided with panel layers wherein one panel layer is folded over another panel layer.

FIG. 4B is a cross-sectional detailed view of a second belt provided with panel layers wherein one panel layer is folded over another panel layer.

FIG. 5A shows a perspective view of a diaper pant with a continuous outer cover in a pre-fastened configuration.

FIG. 5B shows a plan view of a diaper pant with a continuous outer cover with the portion of the diaper that faces away from a wearer oriented toward the viewer.

FIG. 5C is a cross-sectional view of the diaper pant of FIG. 5B taken along line 5C-5C showing first and second elastic belts provided with panel layers and a continuous outer cover.

FIG. 5D is a cross-sectional view of a diaper pant of showing first and second elastic belts provided with panel layers formed with a continuous inner layer and a continuous outer cover.

FIG. 6A is a perspective view of the diaper pant of FIG. 1 showing a first end region of the second belt partially disconnected from a first flange.

FIG. 6B is a perspective view of the diaper pant of FIG. 6A showing a second end region of the second belt partially disconnected from a second flange.

FIG. 7A is a detailed view of a diaper pant showing a flange bonded with a first belt and refastenably connected with a second belt.

FIG. 7B is a detailed view of the diaper pant of FIG. 7A showing the second belt partially disconnected from the flange.

FIG. 7C is a detailed view of the diaper pant of FIG. 7B showing the second belt completely disconnected from the flange.

FIG. 8 is a planar view of a fastener component on a flange.

FIG. 9A is a cross-sectional view of an example configuration of the flange and fastener component of FIG. 8 taken along line 9-9 showing a base of the fastener component bonded with the flange.

FIG. 9B is a cross-sectional view of an example configuration of the flange and fastener component of FIG. 8 taken along line 9-9 showing a base of the fastener component extrusion bonded with a backing layer that is bonded with the flange.

FIG. 9C is a cross-sectional view of an example configuration of the flange and fastener component of FIG. 8 taken along line 9-9 showing a base of the fastener component extrusion bonded with the flange.

FIG. 9D is a cross-sectional view of an example configuration of the flange and fastener component of FIG. 8 taken along line 9-9 showing a base of the fastener component extrusion bonded with the flange.

FIG. 9E is a cross-sectional view of an example configuration of the flange and fastener component of FIG. 8 taken along line 9-9 showing a fastener component comprising hooks formed directly from material of the flange.

FIG. 10A is a detailed view of a diaper pant showing a belt directly refastenably connected with a fastener component on a flange.

FIG. 10B is a detailed view of a diaper pant showing a fastener component on a belt directly refastenably connected with a flange.

FIG. 10C is a detailed view of a diaper pant showing a fastener component comprising loops on a belt refastenably connected with a fastener component comprising hooks on a flange.

FIG. 10D is a detailed view of a diaper pant showing a fastener component comprising hooks on a belt refastenably connected with a fastener component comprising loops on a flange.

FIG. 11 is a side plan view of diaper pant in a flat uncontracted condition showing the garment facing surface, illustrating example elastic material arrangements in the first and second belts.

FIG. 12 is a side plan view of a first belt with a fastener component positioned on a flange prior to engagement with a second belt, illustrating the positioning of the fastener component in relation to the elastic strands.

FIG. 13 is a front plan view of a diaper pant showing the garment facing surface, illustrating various positional relationships with chassis and belt attachment zones and belt elastic zones.

FIG. 14 is a schematic view of an example of a hanger-type sample holding fixture according to the “Whole Article Force Measurement”.

### DETAILED DESCRIPTION

### Definitions

The following term explanations may be useful in understanding the present disclosure:

“Absorbent article” refers to devices, which absorb and contain body exudates and, more specifically, refers to devices, which are placed against or in proximity to the body of the wearer to absorb and contain the various exudates discharged from the body. Exemplary absorbent articles include diapers, training pants, pull-on pant-type diapers (i.e., a diaper having a pre-formed waist opening and leg openings such as illustrated in U.S. Pat. No. 6,120,487), refastenable diapers or pant-type diapers, incontinence briefs and undergarments, diaper holders and liners, feminine hygiene garments such as panty liners, absorbent inserts, menstrual pads and the like.

“Body-facing” and “garment-facing” refer respectively to the relative location of an element or a surface of an element or group of elements. “Body-facing” (also referred to herein as wearer facing) implies the element or surface is nearer to the wearer during wear than some other element or surface. “Garment-facing” implies the element or surface is more remote from the wearer during wear than some other element or surface (i.e., element or surface is proximate to the wearer's garments that may be worn over the disposable absorbent article).

The terms “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force. Elastomeric materials may include elastomeric films, scrims, nonwovens, ribbons, strands and other sheet-like structures.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “distal” is used to describe a position situated away from a center of a body or from a point of attachment, and the term “proximal” is used to describe a position situated nearer to a center of a body or a point of attachment.

The term “substrate” is used herein to describe a material which is primarily two-dimensional (i.e., in an XY plane) and whose thickness (in a Z direction) is relatively small (i.e., 1/10 or less) in comparison to its length (in an X direction) and width (in a Y direction). Non-limiting examples of substrates include a web, layer or layers or fibrous materials, nonwovens, films and foils such as polymeric films or metallic foils. These materials may be used alone or may comprise two or more layers laminated together. As such, a web is a substrate.

The term “nonwoven” refers herein to a material made from continuous (long) filaments (fibers) and/or discontinuous (short) filaments (fibers) by processes such as spunbonding, meltblowing, carding, and the like. Nonwovens do not have a woven or knitted filament pattern.

The term “machine direction” (MD) is used herein to refer to the direction of material flow through a process. In addition, relative placement and movement of material can be described as flowing in the machine direction through a process from upstream in the process to downstream in the process.

The term “cross direction” (CD) is used herein to refer to a direction that is generally perpendicular to the machine direction.

“Pre-strain” refers to the strain imposed on an elastic or elastomeric material prior to combining it with another element of the elastomeric laminate or the absorbent article. Pre-strain is determined by the following equation Pre-strain=((extended length of the elastic-relaxed length of the elastic)/relaxed length of the elastic)*100.

“Decitex” also known as Dtex is a measurement used in the textile industry used for measuring yarns or filaments. 1 Decitex=1 gram per 10,000 meters. In other words, if 10,000 linear meters of a yarn or filament weights 500 grams that yarn or filament would have a decitex of 500.

The term “taped diaper” (also referred to as “open diaper”) refers to disposable absorbent articles having an initial front waist region and an initial back waist region that are not fastened, pre-fastened, or connected to each other as packaged, prior to being applied to the wearer. A taped diaper may be folded about the lateral centerline with the interior of one waist region in surface to surface contact with the interior of the opposing waist region without fastening or joining the waist regions together. Example taped diapers are disclosed in various suitable configurations U.S. Pat. Nos. 5,167,897; 5,360,420; 5,599,335; 5,643,588; 5,674,216; 5,702,551; 5,968,025; 6,107,537; 6,118,041; 6,153,209; 6,410,129; 6,426,444; 6,586,652; 6,627,787; 6,617,016; 6,825,393; and 6,861,571; and U.S. Patent Publication Nos. 2013/0072887 A1; 2013/0211356 A1; and 2013/0306226 A1, all of which are incorporated by reference herein.

The term “pant” (also referred to as “training pant”, “pre-closed diaper”, “diaper pant”, “pant diaper”, and “pull-on diaper”) refers herein to disposable absorbent articles having a continuous perimeter waist opening and continuous perimeter leg openings designed for infant or adult wearers. A pant can be configured with a continuous or closed waist opening and at least one continuous, closed, leg opening prior to the article being applied to the wearer. A pant can be preformed or pre-fastened by various techniques including, but not limited to, joining together portions of the article using any refastenable and/or permanent closure member (e.g., seams, heat bonds, pressure welds, adhesives, cohesive bonds, mechanical fasteners, etc.). A pant can be preformed anywhere along the circumference of the article in the waist region (e.g., side fastened or seamed, front waist fastened or seamed, back waist fastened or seamed). Example diaper pants in various configurations are disclosed in U.S. Pat. Nos. 4,940,464; 5,092,861; 5,246,433; 5,569,234; 5,897,545; 5,957,908; 6,120,487; 6,120,489; 7,569,039 and U.S. Patent Publication Nos. 2003/0233082 A1; 2005/0107764 A1; 2012/0061016 A1; 2012/0061015 A1; 2013/0255861 A1; 2013/0255862 A1; 2013/0255863 A1; 2013/0255864 A1; and 2013/0255865 A1, all of which are incorporated by reference herein.

“Closed-form” means opposing waist regions are joined, as packaged, either permanently or refastenably to form a continuous waist opening and leg openings.

“Open-form” means opposing waist regions are not initially joined to form a continuous waist opening and leg openings but comprise a closure means such as a fastening system to join the waist regions to form the waist and leg openings before or during application to a wearer of the article.

“Inboard,” with respect to a first feature of an article and its position relative a second feature or location on the article, means that the first feature lies closer to a respective axis of the article than the second feature or location, along a horizontal x-y plane approximately occupied by the article when laid out flat, extended to the full longitudinal and lateral dimensions of its component web materials against any contraction induced by any included pre-strained elastomeric material, on a horizontal surface. Laterally inboard means the first feature is closer to the longitudinal axis, and longitudinally inboard means the first feature is closer to the lateral axis. Conversely, “outboard,” with respect to a first feature of an article and its position relative a second feature or location on the article, means that the first feature lies farther from the respective axis of the article than the second feature or location.

The present disclosure relates to absorbent articles, and more particularly, to absorbent articles having refastenably connected front and back waist regions. In some configurations, an absorbent article may comprise: a chassis comprising a topsheet, a backsheet, and an absorbent core positioned between the topsheet and the backsheet. The chassis may further comprise a first end region and a second end region longitudinally separated from the first end region by a crotch region. The absorbent article may comprise a first belt and a second belt, each belt comprising a garment facing surface and an opposing wearer facing surface and each belt comprising a first end region and a second end region laterally separated from the first end region by a central region. The first end region of the chassis may be connected with the central region of the first belt, and the second end region of the chassis may be connected with the central region of the second belt. Each belt may further comprise an outer laterally extending edge and a longitudinally opposed inner laterally extending edge. The absorbent article may also comprise a first flange and a second flange, each flange comprising a first surface and a second surface opposite the first surface, and each flange comprising a first end region and a second end region. The first surface of the first end region of the first flange is bonded with the wearer facing surface of the first end region of the first belt, and the second surface of the second end region of the first flange is in a facing relationship with the wearer facing surface of the second belt. The second end region of the first flange is refastenably connected with the first end region of the second belt. In addition, the first surface of the first end region of the second flange may be bonded with the wearer facing surface of the second end region of the first belt, and the second surface of the second end region of the second flange is in a facing relationship with the wearer facing surface of the second belt. The second end region of the second flange is refastenably connected with the second end region of the second belt.

A portion of the chassis that overlaps the wearer facing surface of the first belt may define a chassis overlap region, wherein the chassis overlap region comprises an attachment area where adhesive is positioned between the chassis and the wearer facing surface of the first belt and permanently bonds the chassis with the first belt. The attachment area may comprise a first attachment zone adjacent the inner laterally extending edge of the first belt and a second attachment zone positioned longitudinally outboard of the first attachment zone. In some configurations, the chassis overlap region may further comprise a first non-bonded region and a second non-bonded region, wherein the first non-bonded region is positioned longitudinally inboard of the second attachment zone and extends laterally between first attachment zone and a first side edge of the chassis, and the second non-bonded region is positioned longitudinally inboard of the second attachment zone and extends laterally between the first attachment zone and a second side edge of the chassis. In some configurations, no adhesive connects the chassis with the first belt in the overlap region in the first non-bonded region and the second non-bonded region.

The first and/or second belts may comprise elastic zones configured to provide a force profile that helps to reduce the tension along the inner laterally extending edge of the first and/or second belt while still providing good fit and comfort of the absorbent article. As discussed below, the arrangements of adhesive and/or elastic zones herein may be configured to help ensure the belt does not curl inward towards the thigh during use, thus helping to prevent the fastener components from coming into contact with the wearer's skin.

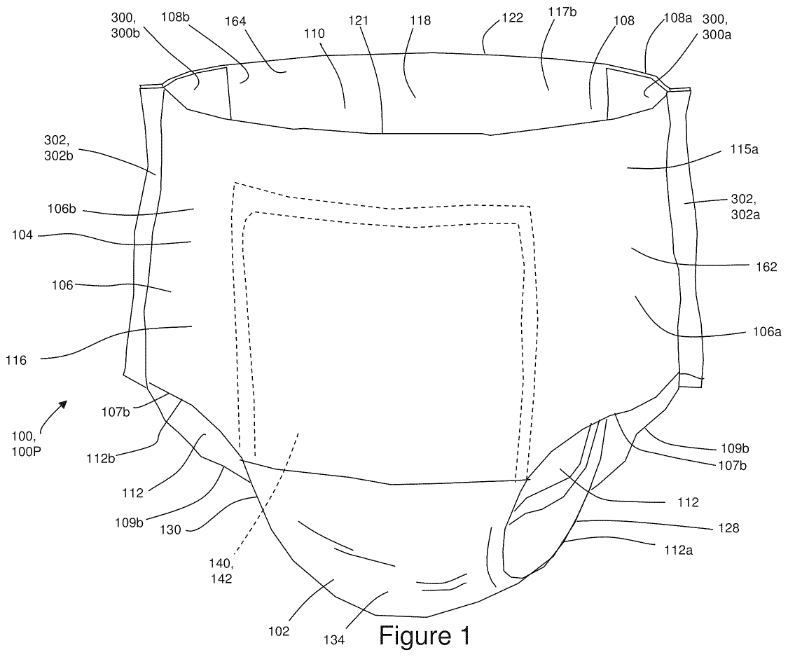

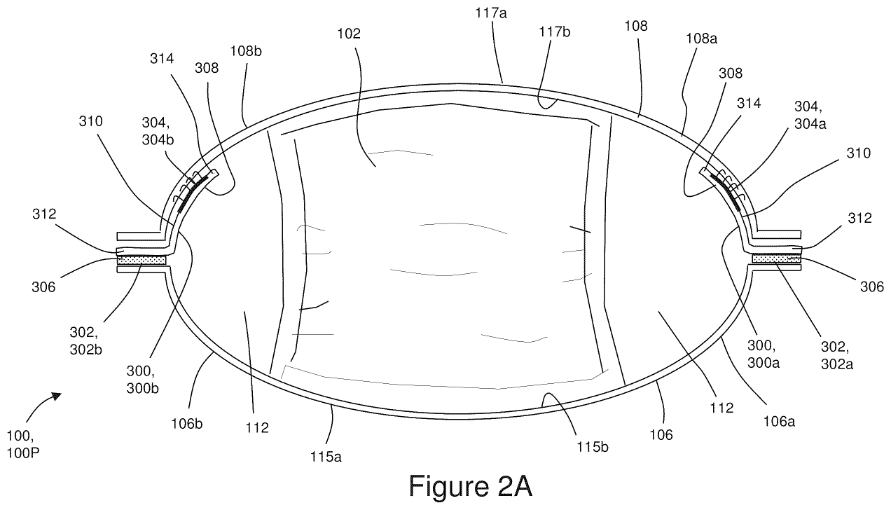

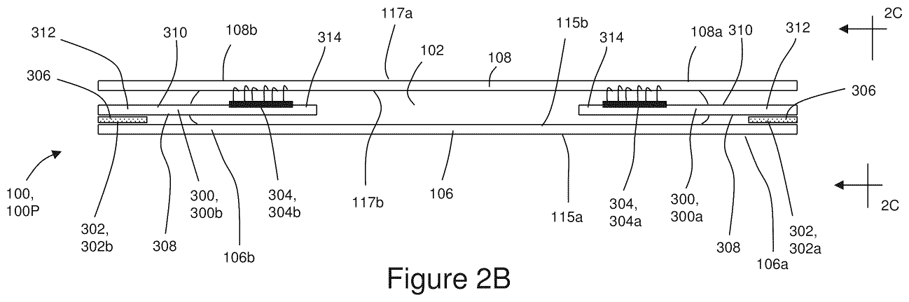

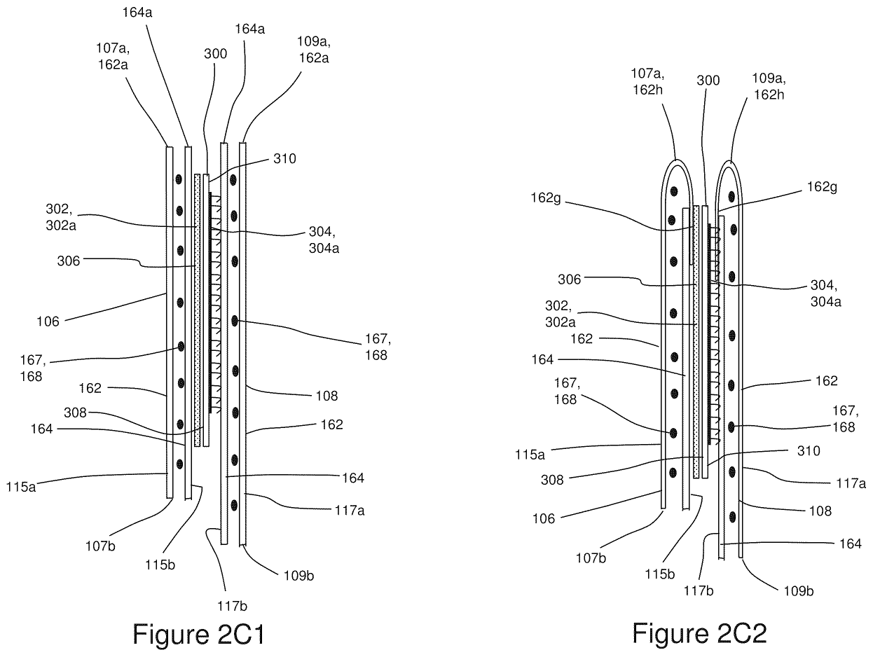

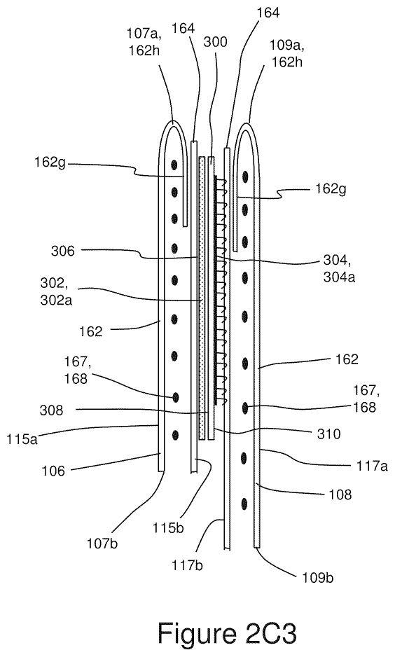

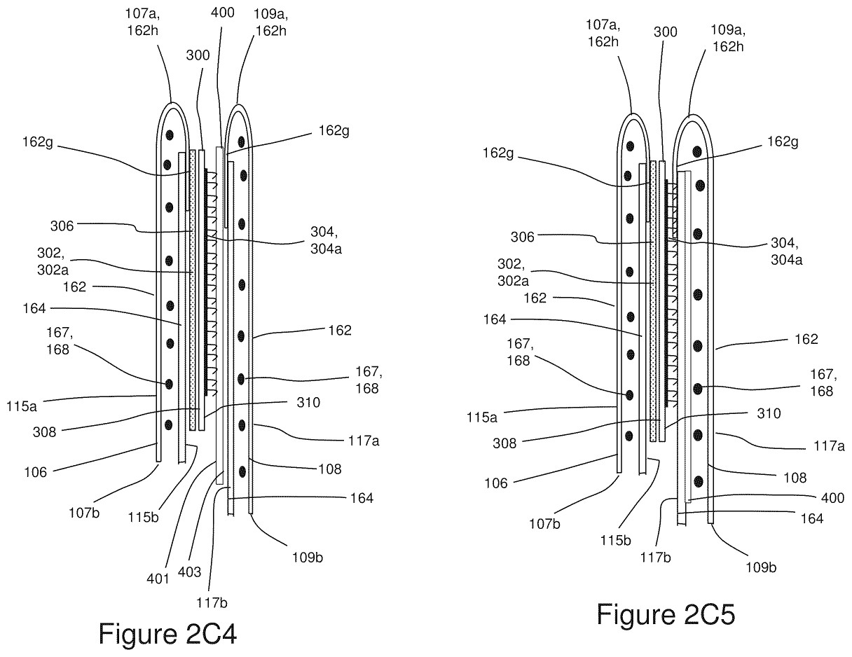

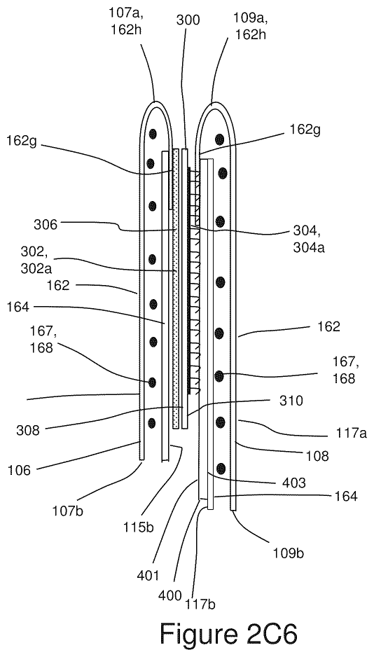

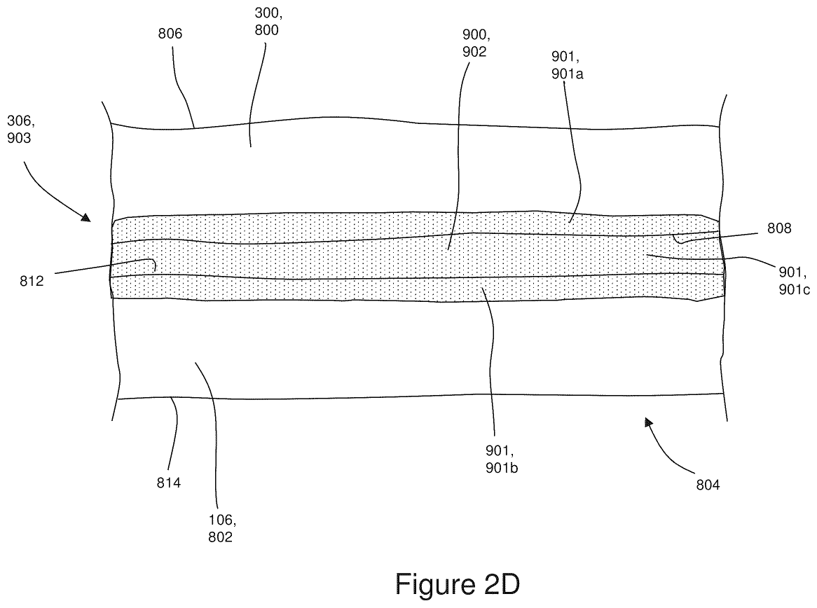

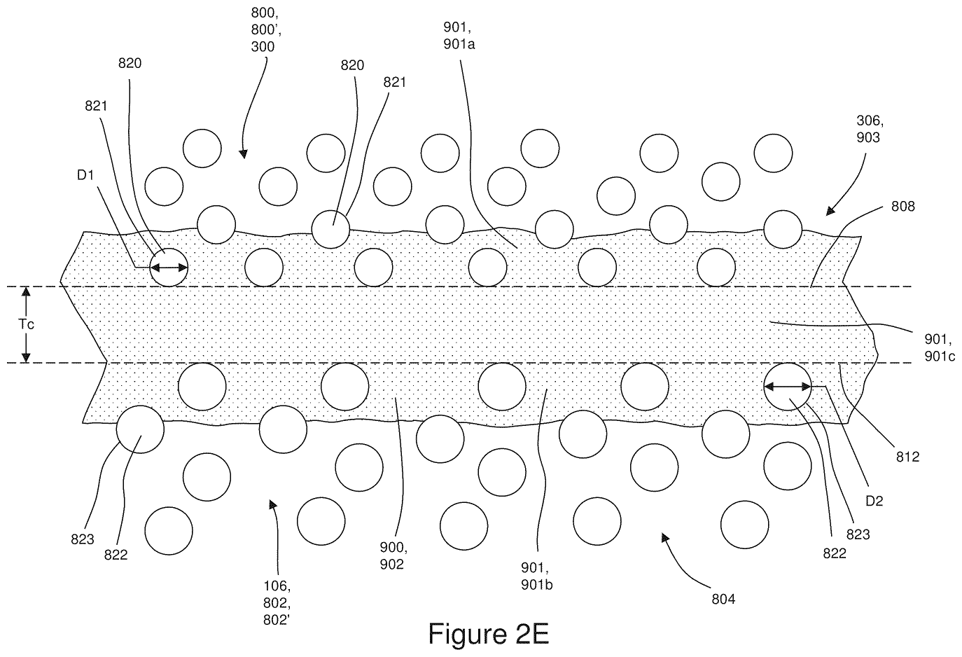

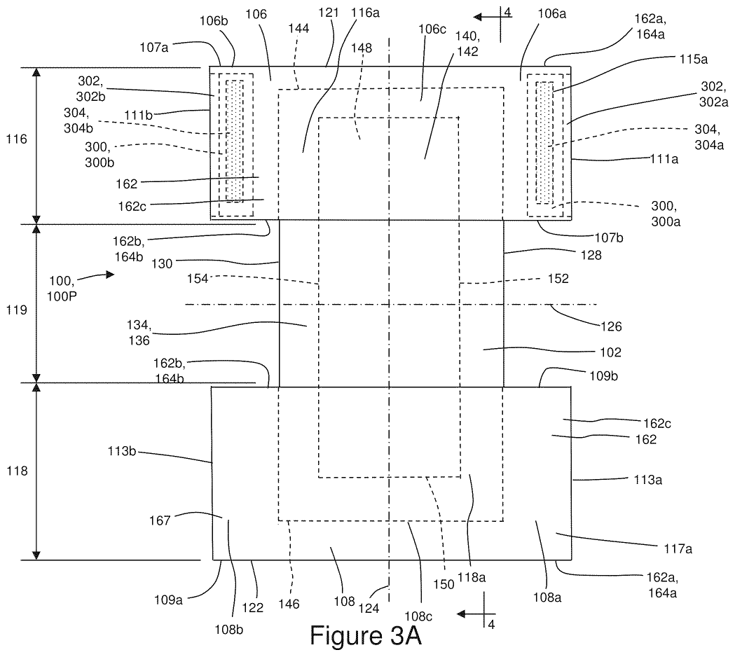

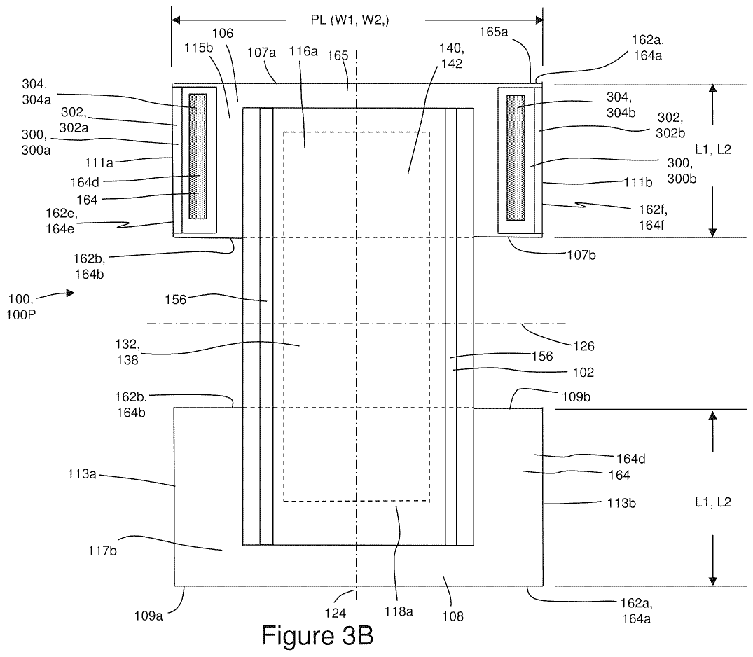

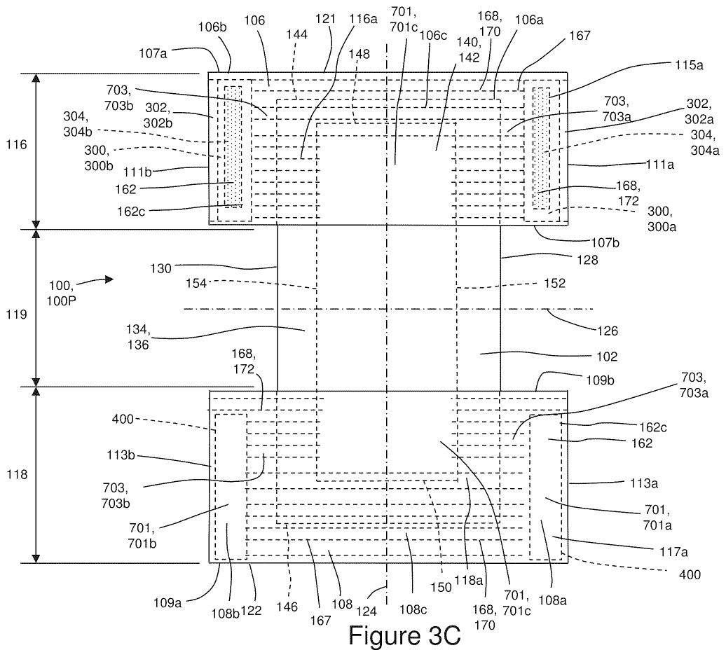

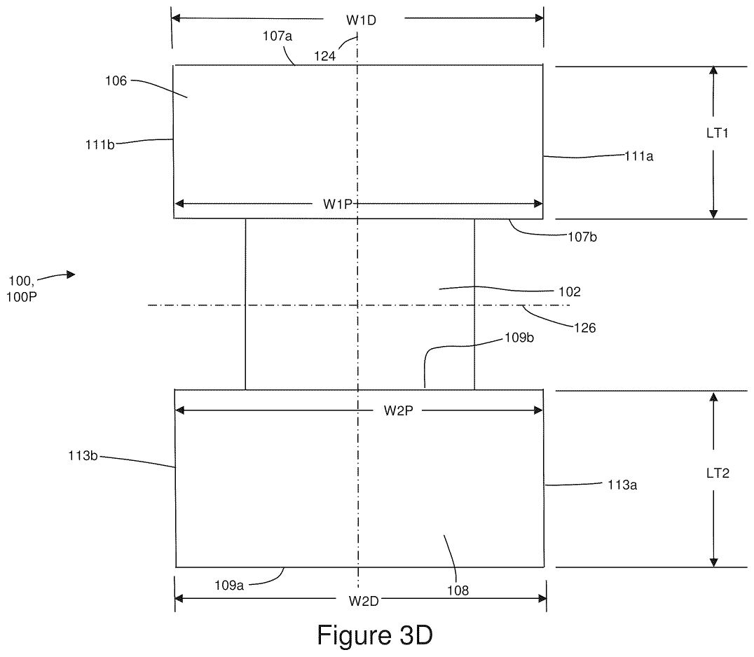

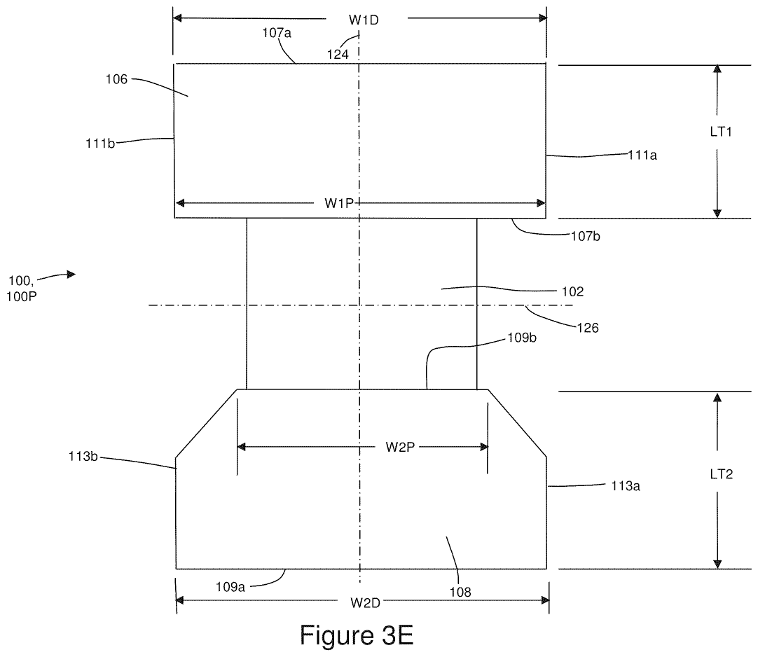

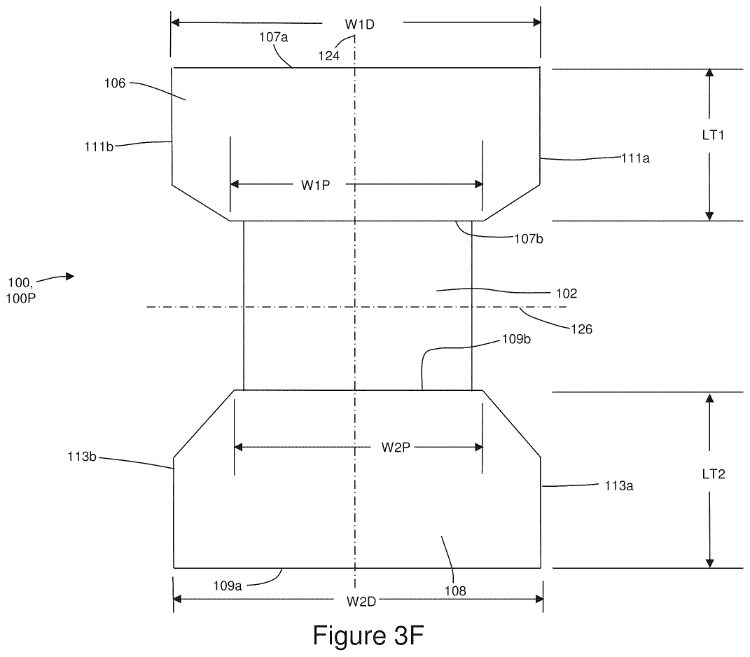

FIGS. 1-3B show an example of an absorbent article 100 in the form of a diaper pant 100P that may include components constructed in accordance with the configurations disclosed herein. In particular, FIG. 1 shows a perspective views of a diaper pant 100P in a pre-fastened configuration. FIG. 2A shows a top view of the diaper pant of FIG. 1, and FIG. 2B shows a top view of the diaper pant of FIG. 2A in a compressed state. FIG. 3A shows a plan view of the diaper pant 100P with the portion of the diaper that faces away from a wearer oriented toward the viewer, and FIG. 3B shows a plan view of the diaper pant 100P with the portion of the diaper that faces toward a wearer oriented toward the viewer. The diaper pant 100P includes a chassis 102 and a ring-like elastic belt 104. As discussed below in more detail, a first elastic belt 106 (also referred to herein as first belt) and a second elastic belt 108 (also referred to herein as second belt) are refastenably connected together to form the ring-like elastic belt 104.

With continued reference to FIGS. 1-3B, the diaper pant 100P and the chassis 102 each include a first waist region 116, a second waist region 118, and a crotch region 119 disposed intermediate the first and second waist regions. It may also be described that the chassis 102 includes a first end region 116a, a second end region 118a, and a crotch region 119 disposed intermediate the first and second end regions 116a, 118a. The first waist region 116 may be configured as a front waist region, and the second waist region 118 may be configured as back waist region. The diaper 100P may also include a laterally extending front waist edge 121 (also referred to herein as a first waist edge) in the first waist region 116 and a longitudinally opposing and laterally extending back waist edge 122 (also referred to herein as a second waist edge) in the back waist region 118 (also referred to as a second waist region). To provide a frame of reference for the present discussion, the diaper 100P and chassis 102 of FIGS. 3A and 3B are shown with a longitudinal axis 124 and a lateral axis 126. In some configurations, the longitudinal axis 124 may extend through the front waist edge 121 and through the back waist edge 122. And the lateral axis 126 may extend through a first longitudinal or right side edge 128 and through a second longitudinal or left side edge 130 of the chassis 102. As previously mentioned, the longitudinal axis 124 extends perpendicularly through the front waist edge 121 and the back waist edge 122, and the lateral axis 126 extends perpendicularly to the longitudinal axis 124. When the diaper pant 100P is worn, the longitudinal direction may extend from the wearer's front waist, through the crotch, to the wearer's back waist.

As shown in FIGS. 1-3B, the diaper pant 100P may include an inner, body facing surface 132, and an outer, garment facing surface 134. The chassis 102 may include a backsheet 136 and a topsheet 138. The chassis 102 may also include an absorbent assembly 140, including an absorbent core 142, disposed between a portion of the topsheet 138 and the backsheet 136. As discussed in more detail below, the diaper 100P may also include other features, such as leg elastics and/or leg cuffs to enhance the fit around the legs of the wearer.

As shown in FIG. 3A, the periphery of the chassis 102 may be defined by the first longitudinal side edge 128, a second longitudinal side edge 130, a first laterally extending end edge 144 disposed in the first waist region 116, and a second laterally extending end edge 146 disposed in the second waist region 118. Both side edges 128 and 130 extend longitudinally between the first end edge 144 and the second end edge 146. As shown in FIG. 3A, the laterally extending end edges 144 and 146 may be located longitudinally inward from the laterally extending front waist edge 121 in the first waist region 116 and the laterally extending back waist edge 122 in the second waist region 118. In some configurations, the laterally extending end edges 144 and 146 may be coterminous with or located longitudinally outward from the laterally extending front waist edge 121 in the first waist region 116 and the laterally extending back waist edge 122 in the second waist region 118. When the diaper pant 100P is worn on the lower torso of a wearer, the front waist edge 121 and the back waist edge 122 may encircle a portion of the waist of the wearer. At the same time, the side edges 128 and 130 may encircle at least a portion of the legs of the wearer. And the crotch region 119 may be generally positioned between the legs of the wearer with the absorbent core 142 extending from the first waist region 116 through the crotch region 119 to the second waist region 118.

As previously mentioned, the diaper pant 100P may include a backsheet 136. The backsheet 136 may also define the outer, garment facing surface 134 of the chassis 102. The backsheet 136 may also comprise a woven or nonwoven material, polymeric films such as thermoplastic films of polyethylene or polypropylene, and/or a multi-layer or composite materials comprising a film and a nonwoven material. The backsheet may also comprise an elastomeric film. An example backsheet 136 may be a polyethylene film having a thickness of from about 0.012 mm (0.5 mils) to about 0.051 mm (2.0 mils). Further, the backsheet 136 may permit vapors to escape from the absorbent core (i.e., the backsheet is breathable) while still preventing exudates from passing through the backsheet 136.

Also described above, the diaper pant 100P may include a topsheet 138. The topsheet 138 may also define all or part of the inner, wearer facing surface 132 of the chassis 102. The topsheet 138 may be liquid pervious, permitting liquids (e.g., menses, urine, and/or runny feces) to penetrate through its thickness. A topsheet 138 may be manufactured from a wide range of materials such as woven and nonwoven materials; apertured or hydroformed thermoplastic films; apertured nonwovens, porous foams; reticulated foams; reticulated thermoplastic films; and thermoplastic scrims. Woven and nonwoven materials may comprise natural fibers such as wood or cotton fibers; synthetic fibers such as polyester, polypropylene, or polyethylene fibers; or combinations thereof. If the topsheet 138 includes fibers, the fibers may be spunbond, carded, wet-laid, meltblown, hydroentangled, or otherwise processed as is known in the art. Topsheets 138 may be selected from high loft nonwoven topsheets, apertured film topsheets and apertured nonwoven topsheets. Exemplary apertured films may include those described in U.S. Pat. Nos. 5,628,097; 5,916,661; 6,545,197; and 6,107,539, all of which are incorporated by reference herein.

As mentioned above, the diaper pant 100P may also include an absorbent assembly 140 that is joined to the chassis 102. As shown in FIG. 3A, the absorbent assembly 140 may have a laterally extending front edge 148 in the first waist region 116 and may have a longitudinally opposing and laterally extending back edge 150 in the second waist region 118. The absorbent assembly may have a longitudinally extending right side edge 152 and may have a laterally opposing and longitudinally extending left side edge 154, both absorbent assembly side edges 152 and 154 may extend longitudinally between the front edge 148 and the back edge 150. The absorbent assembly 140 may additionally include one or more absorbent cores 142 or absorbent core layers. The absorbent core 142 may be at least partially disposed between the topsheet 138 and the backsheet 136 and may be formed in various sizes and shapes that are compatible with the diaper. Exemplary absorbent structures for use as the absorbent core of the present disclosure are described in U.S. Pat. Nos. 4,610,678; 4,673,402; 4,888,231; and 4,834,735, all of which are incorporated by reference herein.

Some absorbent core embodiments may comprise fluid storage cores that contain reduced amounts of cellulosic airfelt material. For instance, such cores may comprise less than about 40%, 30%, 20%, 10%, 5%, or even 1% of cellulosic airfelt material. Such a core may comprise primarily absorbent gelling material in amounts of at least about 60%, 70%, 80%, 85%, 90%, 95%, or even about 100%, where the remainder of the core comprises a microfiber glue (if applicable). Such cores, microfiber glues, and absorbent gelling materials are described in U.S. Pat. Nos. 5,599,335; 5,562,646; 5,669,894; and 6,790,798 as well as U.S. Patent Publication Nos. 2004/0158212 A1 and 2004/0097895 A1, all of which are incorporated by reference herein.

As previously mentioned, the diaper 100P may also include elasticized leg cuffs 156. It is to be appreciated that the leg cuffs 156 can be and are sometimes also referred to as leg bands, side flaps, barrier cuffs, elastic cuffs or gasketing cuffs. The elasticized leg cuffs 156 may be configured in various ways to help reduce the leakage of body exudates in the leg regions. Example leg cuffs 156 may include those described in U.S. Pat. Nos. 3,860,003; 4,909,803; 4,695,278; 4,795,454; 4,704,115; 4,909,803; and U.S. Patent Publication No. 2009/0312730 A1, all of which are incorporated by reference herein.

As mentioned above, diaper pants may be manufactured with a ring-like elastic belt 104 and provided to consumers in a configuration wherein the first waist region 116 and the second waist region 118 are connected to each other as packaged, prior to being applied to the wearer. As such, diaper pants may have a continuous perimeter waist opening 110 and continuous perimeter leg openings 112 such as shown in FIG. 1. The ring-like elastic belt may be formed by joining a first elastic belt to a second elastic belt with an openable and reclosable fastening system disposed at or adjacent the laterally opposing sides of the belts.

As previously mentioned, the ring-like elastic belt 104 may be defined by a first elastic belt 106 connected with a second elastic belt 108. As shown in FIGS. 3A and 3B, the first elastic belt 106 extends between a first longitudinal side edge 111a and a second longitudinal side edge 111b and defines first and second opposing end regions 106a, 106b and a central region 106c. And the second elastic 108 belt extends between a first longitudinal side edge 113a and a second longitudinal side edge 113b and defines first and second opposing end regions 108a, 108b and a central region 108c. As measured in an extended state, the distance between the first longitudinal side edge 111a and the second longitudinal side edge 111b defines the pitch length, PL, of the first elastic belt 106, and the distance between the first longitudinal side edge 113a and the second longitudinal side edge 113b defines the pitch length, PL, of the second elastic belt 108. The central region 106c of the first elastic belt is connected with the first waist region 116 or first end region 116a of the chassis 102, and the central region 108c of the second elastic belt 108 is connected with the second waist region 118 or second end region 118a of the chassis 102.

As shown in FIGS. 1-2B, flanges 300 bonded with opposing end regions of the first belt 106 are refastenably connected with opposing end regions of the second belt 108 to define the ring-like elastic belt 104 as well as the waist opening 110 and leg openings 112. For example, a first flange 300a may be bonded with the first end region 106a of the first belt 106 at a first flange seam 302a, and a second flange 300b may be bonded with the second end region 106b of the first belt 106 at a second flange seam 302b. In turn, the first flange 300a may be refastenably connected with the first end region 108a of the second belt 108, and the second flange 300b may be refastenably connected with the second end region 108b of the second belt 108. For example, as shown in FIGS. 1-2B, a first fastener component 304a on the first flange 300a may refastenably connect the first flange 300a with the second belt 108, and a second fastener component 304b on the second flange 300b may refastenably connect the second flange 300b with the second belt 108. In the configurations shown in FIGS. 2A and 2B, the fastener components 304 may be adapted to refastenably connect directly with the second belt 108. It is to be appreciated that various configurations of fastener components 304 may be located on the flanges 300, the first belt 106, and/or the second belt 108, as discussed in more detail below. It is also to be appreciated that in some configurations, flanges 300 may be bonded with the opposing end regions of the second belt 108 and may be adapted to refastenably connect with opposing end regions of the first belt 106. It is further to be appreciated that the first belt may be positioned in a front waist region or a back waist region, and the second belt may be positioned in a front waist region or a back waist region.

It is also to be appreciated that the fastener components 304 may be configured in various ways, such as hooks, loops, and/or adhesive. For example, the fastener components 304 may comprise hook elements or adhesive adapted to refastenably connect with another surface of the diaper pant 100P. In some configurations, the fastener component 304 may comprise loop elements adapted to refastenably connect with a hook surface on the diaper pant 100P. The fastener component 304 may be a separate element connected with the first belt 106, the second belt 108, and/or the flange 300 in various ways, such as mechanical bonding, adhesive bonding, or both. In some configurations, the fastener component 304 may be integrally formed from materials of the first belt 106, the second belt 108, and/or the flange 300. In some configurations, the flange 300 and/or fastener component 304 may be printed and/or comprise materials of various different colors to help enhance visibility from outside the diaper pant 100P.

It is to be appreciated that the flanges 300 may be constructed from various types of materials, such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some configurations, the flanges 300 may comprise various types of nonwovens, such as spunbond, carded, wet-laid, meltblown, hydroentangled. The flanges 300 may be configured to be stretchable or non-stretchable and/or hydrophilic or hydrophobic. In some configurations, the flanges 300 may be configured as a single layer of material or a laminate comprising two or more layers of material.

It is also to be appreciated that various types of bonds 306 such as illustrated in FIGS. 2A and 2B may be used to bond the flanges 300 with the first belt 106 at the flange seams 302. For example, the bonds 306 at the flange seams 302 may comprise mechanical, thermal, pressure, and/or adhesive bonds.

In some configurations, the bonds 306 may comprise a bond structure 403 that comprises substantially tackifier free adhesives or tackifier free adhesives, such as disclosed in U.S. Patent Publication No. US20200047420A1, which is incorporated by reference herein. The term “tackifier free adhesive” is used herein to refer to an adhesive composition comprising a polymer and/or a copolymer, wherein the adhesive composition is free of or devoid of tackifiers. Examples of such tackifier free adhesives are disclosed in U.S. Patent Publication Nos. US20190322900A1; US20190322901A1; US20190322909A1; US20190321241A1; US20190321242A1; and US20200108167A1, all of which are incorporated by reference herein. “Devoid of,” “free of,” and the like, as those terms are used herein, means that the adhesive composition does not have more than trace amounts or background levels of a given material, ingredient, or characteristic following these qualifiers; the amount of the material or ingredient does not cause harm or irritation that consumers typically associate with the material or ingredient; or the material or ingredient was not added to the adhesive composition intentionally. In some applications, “devoid of” and “free of” can mean there is no measurable amount of the material or ingredient. For example, the adhesive composition in some forms can contain no measurable amount of a tackifier. “Substantially tackifier free adhesive” is used herein to refer to an adhesive composition comprising a polymer and/or a copolymer, wherein the adhesive composition comprises less than 10% tackifiers by weight. As such, a “tackifier free adhesive” is also a “substantially tackifier free adhesive.” The term “tackifier” means those conventional tackifier resins commonly available in the adhesive art and industry that are used in typical hot melt adhesives. Examples of conventional tackifier resins include aliphatic hydrocarbon resins, aromatic modified aliphatic hydrocarbon resins, hydrogenated poly-cyclopentadiene resins, poly-cyclopentadiene resins, gum rosins, gum rosin esters, wood rosins, wood rosin esters, tall oil rosins, tall oil rosin esters, poly-terpene, aromatic modified poly-terpene, terpene-phenolic, aromatic modified hydrogenated poly-cyclopentadiene resins, hydrogenated aliphatic resins, hydrogenated aliphatic aromatic resins, hydrogenated terpene and modified terpene, and hydrogenated rosin esters.

When the flange 300 and the first belt 106 include nonwoven layers, penetration of the substantially tackifier free adhesive into the nonwovens may cause the substantially tackifier free adhesive to intermesh with and bond with fibers within the nonwovens to help strengthen bonds therebetween. FIG. 2D illustrates an example of a bond 306 comprising a bond structure 903 that comprises a substantially tackifier free adhesive 900 between a first substrate 800 of the flange 300 a second substrate 802 of the first belt 106. In particular, FIG. 2D shows an example of a detailed sectional view of a layer 901 of substantially tackifier free adhesive 900 after pressure has been exerted on the first and second substrates 800, 802 to form the bond structure 903 between the first and second substrates 800, 802. As shown in FIG. 2D, the bond 903 comprises a first portion 901a of the layer 901 of substantially tackifier free adhesive 900 that has penetrated into the first substrate 800, and a second portion 901b of the layer 901 of substantially tackifier free adhesive 900 that has penetrated into the second substrate 802. In particular, the first portion 901a of the layer 901 substantially tackifier free adhesive 900 has penetrated through a second surface 808 of first substrate 800 without reaching or exiting a first surface 806 of the first substrate 800. And the second portion 901b of the layer 901 of the substantially tackifier free adhesive 900 has penetrated through a first surface 812 of the second substrate 802 without reaching or exiting a second surface 814 of the second substrate 802. As such, the first portion 901a of the layer 901 of the substantially tackifier free adhesive 900 does not penetrate entirely through the thickness of the first substrate 800, and the second portion 901b of the layer 901 of the substantially tackifier free adhesive 900 does not penetrate entirely through the thickness of the second substrate 802. With continued reference to FIG. 2D, the bond 903 also comprises a central portion 901c of the layer 901, wherein the layer 901 of the substantially tackifier free adhesive 900 comprises a central portion 901c extends between the first portion 901a and the second portion 901b. As illustrated, the second surface 808 of the first substrate 800 and the first surface 812 of the second substrate 802 are separated from each other by the central portion 901c of the layer 901 of the substantially tackifier free adhesive 900.

As previously mentioned, the first substrate 800 may comprise a first nonwoven 800′ and/or the second substrate 802 may comprise a second nonwoven 802′ such as shown in FIG. 2E, wherein the portions of the bond 903 comprises the substantially tackifier free adhesive 900 that is intermeshed with fibers of the first nonwoven 800′ and the second nonwoven 802′. As shown in FIG. 2E, the first nonwoven 800′ may comprise first fibers 820, and the second nonwoven comprises second fibers 822. The first portion 901a of the layer 901 of substantially tackifier free adhesive 900 is intermeshed with the first fibers 820 at the second surface 808 of the first nonwoven 800′, and the second portion 901b of the layer 901 of the substantially tackifier free adhesive 900 is intermeshed with the second fibers 822 at first surface 812 of the second nonwoven 802′. As shown in FIG. 2E, one or more first fibers 820 at and/or adjacent the second surface 808 of the first nonwoven 800′ may comprise an outer perimeter 821 that is completely surrounded by the substantially tackifier free adhesive 900 of the first zone 901a of the layer 901. And one or more second fibers 822 at and/or adjacent the first surface 812 of the second nonwoven 802′ may comprise an outer perimeter 823 that is completely surrounded by the substantially tackifier free adhesive 900 of the second zone 901b of the layer 901. It is to be appreciated that the substantially tackifier free adhesive 900 may completely surround the outer perimeters 821 of additional first fibers 820 positioned away from the second surface 808 and further into the interior thickness of the first nonwoven 800′, and/or the substantially tackifier free adhesive 900 may completely surround the outer perimeters 823 of additional second fibers 822 positioned away from the first surface 812 and further into the interior thickness of the second nonwoven 802′. It is to be appreciated that the substantially tackifier free adhesive 900 may partially surround the outer perimeters 821 of additional first fibers 820 positioned away from the second surface 808 and further into the interior thickness of the first nonwoven 800′, and/or the substantially tackifier free adhesive 900 may partially surround outer perimeters 823 of additional second fibers 822 positioned away from the first surface 812 and further into the interior thickness of the second nonwoven 802′.

With continued reference to FIG. 2E, the first fibers 820 may comprise first diameters D1, and the second fibers 822 may comprise second diameters D2. The first diameters D1 may be the same or different than the second diameters D1. It is to be appreciated that the central portion 901c of the layer 901 of substantially tackifier free adhesive 900 may comprise a thickness Tc. In some configurations, the thickness Tc is greater than the first diameters D1 and/or the second diameters D2. In some configurations, the thickness Tc may be greater than 3 times the first diameters D1 and/or 3 times the second diameters D2.

Referring now to FIGS. 3A and 3B, the first elastic belt 106 also defines an outer laterally extending edge 107a and an inner laterally extending edge 107b, and the second elastic belt 108 defines an outer laterally extending edge 109a and an inner laterally extending edge 109b. The outer edge 107a of the first belt 106 is positioned longitudinally outward of the inner edge 107b, and the outer edge 109a of the second belt 108 is positioned longitudinally outward of the inner edge 109b. As such, as shown in FIG. 1, a perimeter edge 112a of one leg opening may be defined by portions of the inner laterally extending edge 107b of the first elastic belt 106, the inner laterally extending edge 109b of the second elastic belt 108, and the first longitudinal or right side edge 128 of the chassis 102. And a perimeter edge 112b of the other leg opening may be defined by portions of the inner laterally extending edge 107b, the inner laterally extending edge 109b, and the second longitudinal or left side edge 130 of the chassis 102. The outer laterally extending edges 107a, 109a may also define the front waist edge 121 and the laterally extending back waist edge 122 of the diaper pant 100P.

It is to be appreciated that the first elastic belt 106 and the second elastic belt 108 may define different sizes and shapes. In some configurations, the first elastic belt 106 and/or second elastic belt 108 may define curved contours. For example, the inner lateral edges 107b, 109b of the first and/or second elastic belts 106, 108 may include non-linear or curved portions in the first and second opposing end regions. Such curved contours may help define desired shapes to leg opening 112, such as for example, relatively rounded leg openings. In addition to having curved contours, the elastic belts 106, 108 may include elastic strands 168 that extend along non-linear or curved paths that may correspond with the curved contours of the inner lateral edges 107b, 109b.

FIG. 3D shows a configuration wherein the first elastic belt 106 and the second elastic belt 108 both define generally rectangular shapes. For example, as shown in FIG. 3D, the outer laterally extending edge 107a of the first elastic belt 106 may comprise a lateral width of WID and the inner laterally extending edge 107b may comprise a lateral width of W1P, wherein WID and W1P are equal or substantially equal. In addition, the outer laterally extending edge 109a of the second elastic belt 108 may comprise a lateral width of W2D and the inner laterally extending edge 109b may comprise a lateral width of W2P, wherein W2D and W2P are equal or substantially equal.

In some configurations, at least one of the first elastic belt 106 and the second elastic belt 108 may comprise lateral edges having different lengths. For example, FIG. 3E shows a configuration wherein the first elastic belt 106 defines a generally rectangular shape, such as described with reference to FIG. 3D, and wherein the outer laterally extending edge 109a of the second elastic belt 108 and the inner laterally extending edge 109b have different lengths. As shown in FIG. 3E, the outer laterally extending edge 109a of the second elastic belt 108 may comprise a lateral width of W2D and the inner laterally extending edge 109b may comprise a lateral width of W2P, wherein W2D is greater than W2P.

In some configurations, both the first elastic belt 106 and the second elastic belt 108 may comprise lateral edges having different lengths. For example, FIG. 3F shows a configuration wherein the outer laterally extending edge 107a of the first elastic belt 106 and the inner laterally extending edge 107b have different lengths, and wherein the outer laterally extending edge 109a of the second elastic belt 108 and the inner laterally extending edge 109b have different lengths. As shown in FIG. 3F, the outer laterally extending edge 107a of the first elastic belt 107 may comprise a lateral width of W1D and the inner laterally extending edge 107b may comprise a lateral width of W1P, wherein W1D is greater than W1P, and wherein the outer laterally extending edge 109a of the second elastic belt 108 may comprise a lateral width of W2D and the inner laterally extending edge 109b may comprise a lateral width of W2P, wherein W2D is greater than W2P.

With reference to FIGS. 3D-3F, the first elastic belt 106 may define a longitudinal length LT1 extending between outer laterally extending edge 107a and the inner laterally extending edge 107b, and the second elastic belt 108 may define a longitudinal length LT2 extending between outer laterally extending edge 109a and the inner laterally extending edge 109b. In some configurations, LT1 may be equal to LT2. In some configurations, LT1 may be less or greater than LT2. With continued reference to FIGS. 3D-3F, in some configurations, W1D may be equal to W1P, or W1D may be different than W1P. In some configurations, W2D may be equal to W2P, or W2D may be different than W2P. In some configurations, W1D and/or W1P may be equal to or different W2D and/or W2P.

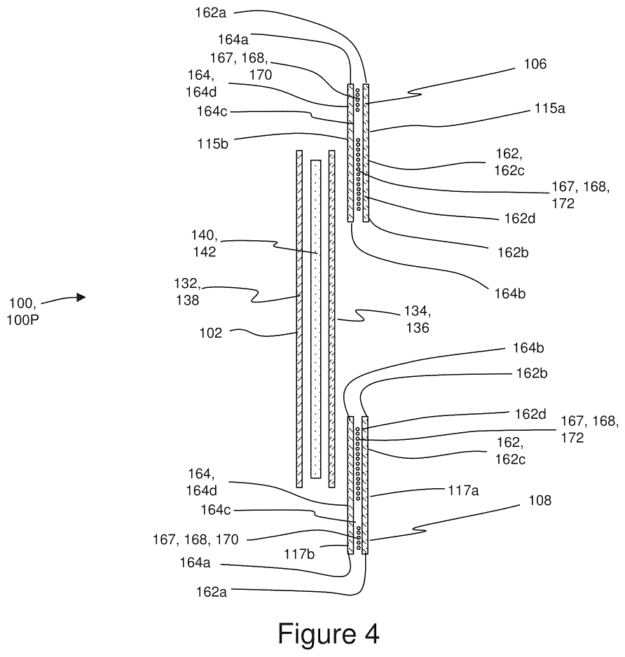

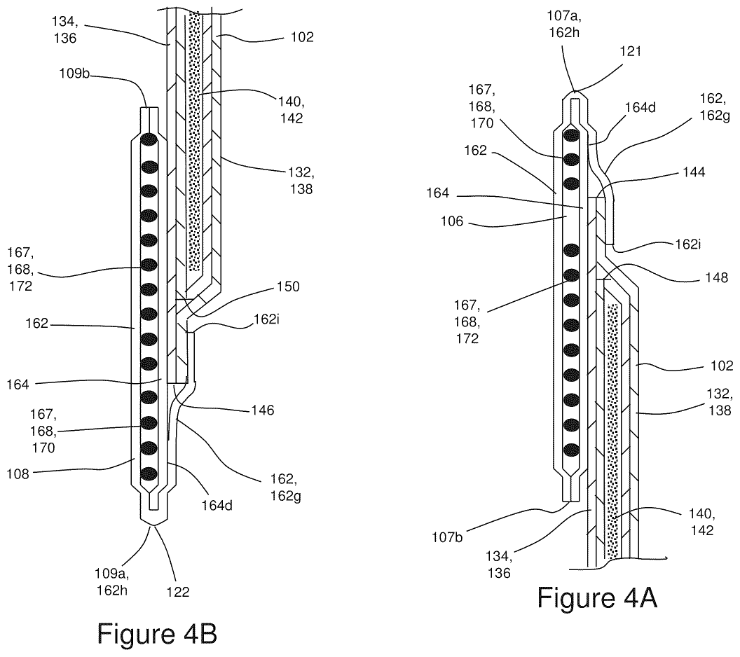

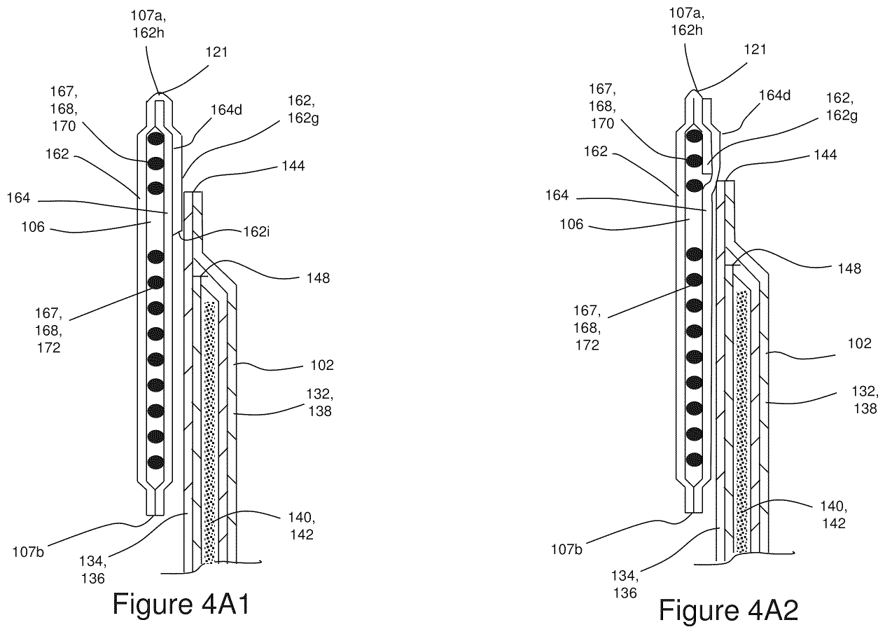

With reference to FIGS. 3A, 3B, and 4, the first elastic belt 106 and the second elastic belt 108 may also each include a first substrate 162 and a second substrate 164. The first substrates 162 may be oriented to define at least a portion of a garment facing surface 115a of the first elastic belt 106 and a garment facing surface 117a of the second elastic belt 108, and the second substrates 164 may be oriented to define at least a portion of a wearer facing surface 115b of the first elastic belt 106 and a wearer facing surface 117b of the second elastic belt 108. The first substrate 162 may extend from a proximal edge 162b to a distal edge 162a for a maximum length L1, and the second substrate 164 may extend from a proximal edge 164b to a distal edge 164a for a maximum length L2. It is to be appreciated that the distal edge 162a and/or the proximal edge 162b of the first substrate 162 may be straight and/or curved and/or may be parallel or unparallel to each other. It is also to be appreciated that the distal edge 164a and/or the proximal edge 164b of the second substrate 164 may be straight and/or curved and/or may be parallel or unparallel to each other. As such, the maximum length L1 refers to the longest distance extending longitudinally between the distal edge 162a and the proximal edge 162b of the first substrate 162, and the maximum length L2 refers to the longest distance extending longitudinally between the distal edge 164a and the proximal edge 164b of the second substrate 164. In some configurations, L1 may be equal to, less than, or greater than L2. In some configurations, L1 may be equal to or less than LT1, and L2 may be equal to or less than LT2. In some configurations, the distal edge 162a of the first substrate 162 may define at least a portion of the front waist edge 121 and/or at least a portion of back waist edge 122, and/or the distal edge 164a of the second substrate 164 may define at least a portion of the front waist edge 121 and/or at least a portion of back waist edge 122. As such, in some configurations, the distal edge 162a of the first substrate 162 and/or the distal edge 164a of the second substrate 164 may define at least a portion of the waist opening 110.

It is to be appreciated that the first substrate 162 and the second substrate 164 may define various lateral widths that may or may not be equal. For example, as shown in FIG. 3B, the first substrate 162 may extend laterally between a first longitudinal edge 162e and a second longitudinal edge 162f to define a first lateral width W1, and the second substrate 164 may extend laterally between a first longitudinal edge 164e and a second longitudinal edge 164f to define a second lateral width W2.

In some configurations, the proximal edge 162b of the first substrate 162 and/or the proximal edge 164b of the second substrate 164 may extend laterally across the backsheet 136. As shown in FIGS. 3A, 3B, and 4, the first substrate 162 includes a garment facing surface 162c and an opposing wearer facing surface 162d, and the second substrate 164 includes a garment facing surface 164c and an opposing wearer facing surface 164d.

In some configurations, the first elastic belt 106 and/or the second elastic belt 108 may include a folded portion of at least the first substrate 162 and/or the second substrate 164. For example, as shown in FIGS. 4A and 4B, the first elastic belt 106 and/or the second elastic belt 108 may include a folded portion 162g of the first substrate 162 extending longitudinally between a fold line 162h in the first substrate 162 and a lateral edge 162i. As such, the folded portion 162g of the first substrate 162 may be connected with the wearer facing surface 164d of the second substrate 164. In some configurations, the folded portion 162g of the first substrate 162 may also be connected with and/or overlap the chassis 102. In some configurations, the folded portion 162g of the first substrate 162 may also be connected with the wearer facing surface 162d of the first substrate 162. In some configurations, a portion of the folded portion 162g of the first substrate 162 may be left unbonded to the chassis 102 and/or the second substrate 164, forming a pocket having an opening oriented toward the lateral centerline 162c of the chassis 102. In another example, the first elastic belt 106 and/or the second elastic belt 108 may include a folded portion of the second substrate 164 extending longitudinally between a fold line in the second substrate 164 and a lateral edge. As such, the folded portion of the second substrate 164 may be connected with the garment facing surface 162c of the first substrate 162. As such, in some configurations, a fold line of the first substrate 162 and/or a fold line of the second substrate 164 may define at least a portion of the waist opening 110. It is to be appreciated that various waist configurations may be utilized. For example, as shown in FIG. 4A1, the folded portion 162g may be sandwiched between the second substrate 164 and the backsheet 136. In another example shown in FIG. 4A2, the second substrate 164 may be sandwiched between the folded portion 162g and the backsheet 136. Although FIGS. 4A1 and 4A2 show configurations of the first belt 106, it is to be appreciated that such configurations may be applied with the second belt 108.

It is to be appreciated that the first elastic belt 106 and the second elastic belt 108 may comprise the same materials and/or may have the same structure. In some embodiments, the first elastic belt 106 and the second elastic belt 108 may comprise different materials and/or may have different structures. It should also be appreciated that components of the first elastic belt 106 and the second elastic belt 108, such as the first substrate 162, and/or second substrate 164 may be constructed from various materials. For example, the first and/or second belts may include a first substrate 162, and/or second substrate 164 that may be manufactured from materials such as plastic films; apertured plastic films; woven or nonwoven webs of natural materials (e.g., wood or cotton fibers), synthetic fibers (e.g., polyolefins, polyamides, polyester, polyethylene, or polypropylene fibers) or a combination of natural and/or synthetic fibers; or coated woven or nonwoven webs. In some configurations, the first and/or second belts may include a first substrate 162, and/or second substrate 164 comprising a nonwoven web of synthetic fibers, and may include a stretchable nonwoven. In some configurations, the first and second elastic belts may include an inner hydrophobic, non-stretchable nonwoven material and an outer hydrophobic, non-stretchable nonwoven material. In some configurations, the first and/or second belts may include a first substrate 162 and/or second substrate 164 comprising a nonwoven material having a basis weight of from about 10 to about 35 gsm, or from about 15 to about 25 gsm. It is to be appreciated that the belts may configured in various ways, such as disclosed for example, in U.S. Patent Publication No. 2022/0142828A1, which is incorporated by reference.

Elastic material 167 may be positioned between the wearer facing surface 162d of the first substrate 162 and the garment facing surface 164c of the second substrate 164. It is to be appreciated that the elastic material 167 may include one or more elastic elements such as strands, ribbons, elastic films, or panels extending along the lengths of the elastic belts. As shown in FIGS. 3C and 4, the elastic material 167 may include a plurality of elastic strands 168. In some configurations, the elastic material 167 may be an elastic film used to form a zero-strain elastic laminate comprising an elastic film bonded to one or more nonwoven layers and subsequently subjected to mechanical deformation or activation sufficient to weaken the nonwoven layer(s) and enable the laminate to stretch and recover elastically.

It is also to be appreciated that the first substrate 162, second substrate 164, and/or elastic material 167 of the first elastic belt 106 and/or second elastic belt 108 may be bonded together and/or with other components, such as the chassis 102, with adhesive and/or mechanical bonds. It is to be appreciated that adhesive and mechanical bonding methods may be utilized alone or in combination with each other.

In some configurations, adhesive may be applied to at least one of the first substrate 162, second substrate 164, and/or elastic material 167 when being combined to form the first elastic belt 106 and/or second elastic belt 108. In some configurations, mechanical bonding devices may apply mechanical bonds to the at least one of the first substrate 162, second substrate 164, and/or elastic material 167 when being combined to form the first elastic belt 106 and/or second elastic belt 108. Such mechanical bonds may be applied with heat, pressure, and/or ultrasonic devices. In some configurations, mechanical bonding devices may apply bonds that bond the first substrate 162, second substrate 164, and/or elastic material 167 together and/or may act to trap or immobilize discrete lengths of the contracted elastic strands in the first elastic belt 106 and/or second elastic belt 108.

It is to be appreciated that components of the first elastic belt 106 and/or the second elastic belt 108 may be assembled in various ways and various combinations to create various desirable features that may differ along the lateral width and/or longitudinal length of the first elastic belt 106 and/or the second elastic belt 108. Such features may include, for example, Dtex values, bond patterns, aperture arrangements, elastic positioning, Average Dtex values, Average Pre-Strain values, rugosity frequencies, rugosity wavelengths, height values, and/or contact area. It is to be appreciated that differing features may be imparted to various components, such as for example, the first substrate 162, second substrate 164, and elastic material 167 before and/or during stages of assembly of the first elastic belt 106 and/or the second elastic belt 108.

It is to be appreciated that the first elastic belt 106 and/or the second elastic belt 108 may include various configurations of belt elastic materials 167 arranged in relation to each other and to the first substrate 162, and the second substrate 164. As discussed above, the elastic material 167 may include configurations of one or more elastic elements such as strands, ribbons, films, or panels positioned in various arrangements. In some configurations, the elastic material 167 may comprise various elastics, elastic features and arrangements, and processes for assembly, such as described in 2018/0168889 A1; 2018/0168874 A1; 2018/0168875 A1; 2018/0168890 A1; 2018/0168887 A1; 2018/0168892 A1; 2018/0168876 A1; 2018/0168891 A1; 2019/0298586 A1; 2019/0070042 A1; 2018/0168878 A1; 2018/0168877 A1; 2018/0168880 A1; 2018/0170027 A1; 2018/0169964 A1; 2018/0168879 A1; 2018/0170026 A1; 2019/0070041 A1; 2021/0282797 A1; and 2021/0275362 A1, which are all incorporated by reference. It is also to be appreciated that the elastic materials 167 herein may be configured with identical or different colors in various different locations on the first elastic belt 106 and/or the second elastic belt 108.

In some configurations, the elastic material 167 may be configured as elastic strands 168 disposed at a constant interval in the longitudinal direction. In other embodiments, the elastic strands 168 may be disposed at different intervals in the longitudinal direction. In some configurations, the Dtex values of the elastic strands 168 may be constant or varied along the longitudinal direction. In some configurations, the elastic material 167 in a stretched condition may be interposed and joined between uncontracted substrate layers. When the elastic material 167 is relaxed, the elastic material 167 returns to an unstretched condition and contracts the substrate layers. The elastic material 167 may provide a desired variation of contraction force in the area of the ring-like elastic belt. It is to be appreciated that the chassis 102 and elastic belts 106, 108 may be configured in different ways other than as depicted in attached Figures. It is also to be appreciated that the elastic material 167 material may be joined to the substrates continuously or intermittently along the interface between the elastic material 167 material and the substrates. In some configurations, the elastic strands 168 may be in the form of extruded elastic strands, which may also be bonded with the first substrate 162 and/or second substrate 164 in a pre-corrugated configuration, such as disclosed for example in U.S. Pat. No. 5,681,302, which is incorporated by reference herein.

As discussed above for example with reference to FIGS. 3C and 4, the elastic material 167 discussed herein may be in the form of elastic strands 168. In some configurations, the elastic strands 168 may be parallel with each other and/or with the lateral axis 126. It is to be appreciated that the first elastic belt 106 and/or second elastic belt 108 may be configured to include various quantities of elastic strands 168. In some configurations, elastic strands 168 may be grouped in pairs. In some configurations, the first elastic belt 106 and/or second elastic belt 108 may comprise from about 10 to about 1500 elastic strands 168.

It is also to be appreciated that elastic strands 168 herein may comprise various Dtex values, strand spacing values, and pre-strain values and such elastic strands 168 may utilized with other elastic strands to create first and second elastic belts 106, 108 comprising elastic strands 168 in various combinations of Dtex values, strand spacing values, and pre-strain values. For example, in some configurations, the Average-Dtex of one or more elastic strands 168 may be greater than 500. In some configurations, the Average-Dtex of one or more elastic strands 168 may be from about 10 to about 1500, specifically reciting all 1 Dtex increments within the above-recited range and all ranges formed therein or thereby. In some configurations, a plurality of elastic strands 168 may comprise an Average-Strand-Spacing of less than or equal to 15 mm. In some configurations, a plurality of elastic strands 168 may comprise an Average-Strand-Spacing of less than or equal to 4 mm. In some configurations, a plurality of elastic strands 168 may comprise an Average-Strand-Spacing from about 0.25 mm to about 15 mm, specifically reciting all 0.01 mm increments within the above-recited range and all ranges formed therein or thereby. In some configurations, a plurality of elastic strands 168 may comprise an Average-Strand-Spacing of greater than 4 mm. In some configurations, the Average-Pre-Strain of each of a plurality of elastic strands may be from about 50% to about 400%, specifically reciting all 1% increments within the above-recited range and all ranges formed therein or thereby. In some configurations, the elastic strands 168 comprise an Average-Strand-Spacing from about 0.25 mm to about 4 mm and an Average-Dtex from about 10 to about 500. In some configurations, the elastic strands 168 may comprise an Average-Strand-Spacing from about 0.25 mm to about 8 mm and an Average-Dtex from about 10 to about 1000. In some configurations, the elastic strands 168 may comprise an Average-Pre-Strain from about 75% to about 300%.

In some configurations, a first plurality of elastic strands may comprise a first Average-Pre-Strain from about 75% to about 300%, and a second plurality of elastic strands may comprise a second Average-Pre-Strain that is greater than first Average-Pre-Strain. In some configurations, a first plurality of elastic strands comprises an Average-Strand-Spacing from about 0.25 mm to about 4 mm and an Average-Dtex from about 10 to about 500; and a second plurality of elastic strands may comprise an Average-Strand-Spacing greater than about 4 mm and an Average-Dtex greater than about 450.

In some configurations, such as shown in FIG. 3C, the elastic strands 168 may be referred to herein as outer waist elastics 170 and inner waist elastics 172. Elastic strands 168, such as the outer waist elastics 170, may continuously extend laterally between the first and second opposing end regions 106a, 106b of the first elastic belt 106 and between the first and second opposing end regions 108a, 108b of the second elastic belt 108. Some elastic strands 168, such as the inner waist elastics 172, may be configured with discontinuities in areas, such as for example, where the first and second elastic belts 106, 108 overlap portions of the chassis 102, such as the absorbent assembly 140. In some configurations, some outer waist elastics 170 and/or inner waist elastics 172 may be configured with discontinuities in areas adapted to refastenably connect with the flanges 300.

As shown in FIG. 3C, the first elastic belt 106 and/or the second elastic belt 108 may be configured with low-stretch zones 701 and high-stretch zones 703. The first elastic belt 106 and/or the second elastic belt 108 may include a first high-stretch zone 703a and a second high-stretch zone 703b separated laterally by a central low-stretch zone 701c. Portions of the chassis 102, such as the backsheet 136 and absorbent assembly 140, may be connected with the first elastic belt 106 and/or the second elastic belt 108 in the central low-stretch zones 701c in the first waist region 116 and/or the second waist region 118. The first elastic belt 106 and/or the second elastic belt 108 may also include a first lateral low-stretch zone 701a and a second lateral low-stretch zone 701b. In some configurations, the second belt 108 may include first and second lateral low-stretch zones 701a, 701b located in areas where flanges 300 may be refastenably connected with the second belt 108. In some configurations, the first and second low-stretch zones 701a, 701b may be positioned adjacent to the outer laterally extending edge 109a of the second belt 108. The first and second low-stretch zones 701a, 701b may have a lateral width of from about 20 mm to about 50 mm, or from about 30 mm to about 40 mm. The first and second low-stretch zones 701a, 701b may have a longitudinal length of from about 110 mm to about 160 mm, or from about 115 mm to about 155 mm, or from about 120 mm to about 150 mm. In some configurations, the longitudinal length of the first and/or second low-stretch zones 701a, 701b may be about 10 mm longer than the longitudinal length of the fastener component 304.

In some configurations, the first and/or second low-stretch zones 701a, 701b of the second belt 108 may comprise a stiffening patch 400 in areas where flanges 300 may be refastenably connected with the second belt 108. Without being limited by theory, it is believed that the stiffening patch 400 may help to increase the stiffness of the first and second end regions of the second belt and aid in fastening (and refastening) of the belts by reducing the collapse of the second belt and/or creating a flatter area that can engage more effectively with the fastener component on the first belt.

The high-stretch zones 703 are elasticated by the elastic material 167, such as the elastic strands 168, 172; and the low-stretch zones 701 may comprise cut lines separating the elastic material 167, such as the elastic strands 168, 172. In some configurations, the elastic material 167 may be cut in an unbonded region where the elastic material is not bonded with first substrate 162 and the second substrate 164. Thus, the elastic material 167 retracts from the unbonded region and form low-stretch zone 701. In some configurations, the elastic material 167 may be cut into several discrete pieces. In turn, the low-stretch zones 701 define regions of the first elastic belt 106 and/or the second elastic belt 108 that have relatively less elasticity than the high-stretch zones 703. The discrete elastic material 167 that has been cut and which are elastically contracted do not add any substantial amount of elastication to the low-stretch zone 701. As such, upon application of a force, the high-stretch zones 703 will elongate more than the low-stretch zones 701. As provided above, the terms “elastic,” “elastomer” or “elastomeric” refers to materials exhibiting elastic properties, which include any material that upon application of a force to its relaxed, initial length can stretch or elongate to an elongated length more than 10% greater than its initial length and will substantially recover back to about its initial length upon release of the applied force. In some configurations, the first elastic belt 106 and/or the second elastic belt 108 may be configured with high-stretch zones 703 that are elastic and may be configured with low-stretch zones 701 that are not elastic or “inelastic.”

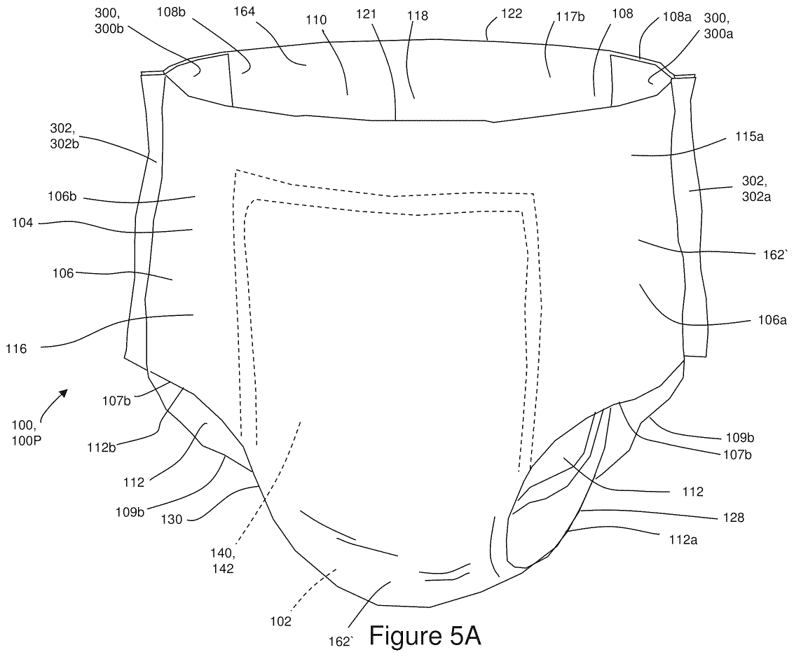

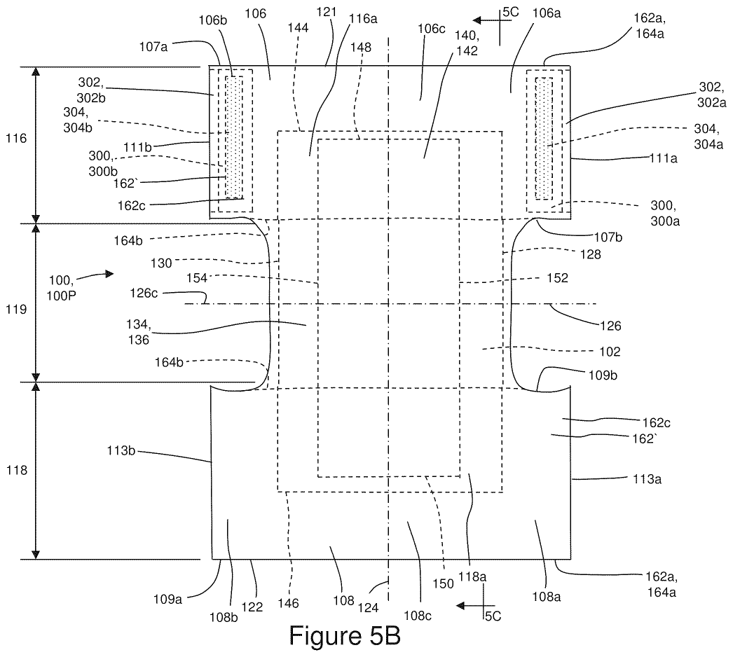

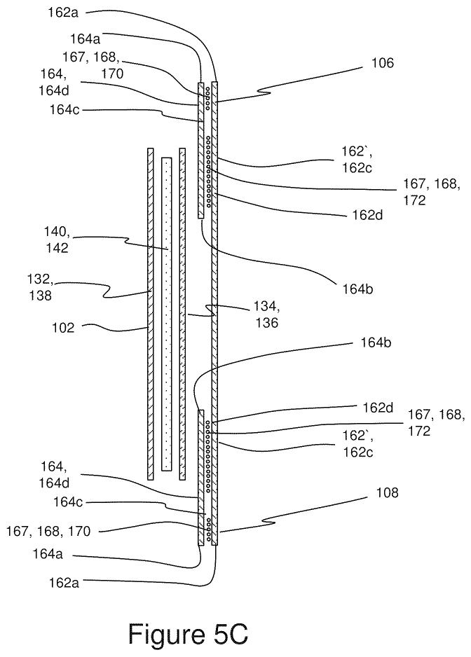

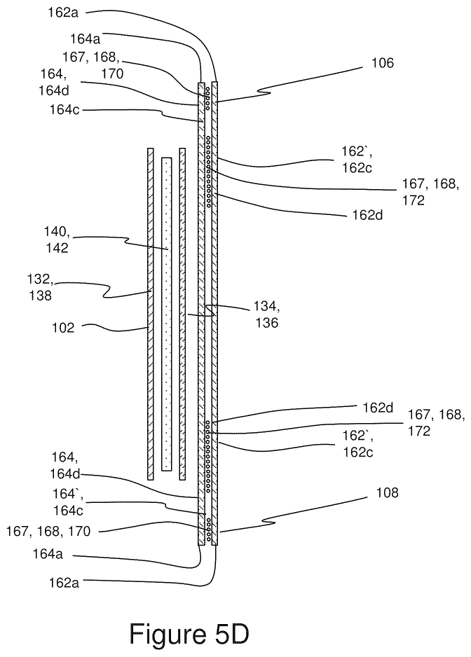

It is also to be appreciated diaper pants 100P may be configured with the first substrate 162 and/or the second substrate 164 that may extend continuously from the first belt 106 to the second belt 108. For example, the first substrate 162 may be configured to define a continuous outer cover 162′ that extends contiguously from the first waist edge 121 to the second waist edge 122, such as shown in FIGS. 5A-5C. FIG. 5D shows a diaper pant 100P with both the first substrate 162 configured to define a continuous outer cover 162′ and the second substrate 164 configured to define a continuous inner layer 164′ that extend contiguously from the first waist edge 121 to the second waist edge 122. It is also to be appreciated that diaper pants 100P with continuous outer covers, such as shown in FIGS. 5A-5D may also be configured to include various aspects of the elastic material 167, flanges 300, and fastener components 304 discussed herein as well as waist edge configurations described about with reference to FIGS. 4 through 4A2.

As discussed above, the diaper pant 100P may include flanges 300 bonded with opposing end regions of the first belt 106, and the flanges 300 may be refastenably connected with opposing end regions of the second belt 108. As shown in FIGS. 2A and 2B, the flanges 300 may each include a first surface 308 and an opposing second surface 310. The flanges 300 may further comprise a first lateral end region 312 and a second lateral end region 314. The first surface 308 of the first lateral end region 312 of the first flange 300a may be bonded with the first end region 106a of the first belt 106 at the first flange seam 302a, and the second surface 310 of the second lateral end region 314 of the first flange 300a may be refastenably connected with the first end region 108a of the second belt 108. In some configurations, a first fastener component 304a on the second surface 310 of the second lateral end region 314 of the first flange 300a may be refastenably connected with first end region 108a of the second belt 108. In addition, the first surface 308 of the first lateral end region 312 of the second flange 300b may be bonded with the second end region 106b of the first belt 106 at the second flange seam 302b, and the second surface 310 of the second lateral end region 314 of the second flange 302b may be refastenably connected with the second end region 108b of the second belt 108. In some configurations, a second fastener component 304b on the second surface 310 of the second lateral end region 314 of the second flange 300a may be refastenably connected with second end region 108b of the second belt 108. In some configurations, the fastener component 304 may be positioned laterally inboard of the flange seam 302. In some configurations, the fastener component 304 and the flange seam 302 may be spaced apart by a distance of from about 3 mm to about 11 mm, or from about 5 mm to about 8 mm.

With reference to FIG. 2B, when the diaper pant 100P is in a compressed, folded state, such as when placed in a package, with the wearer facing surface 115b of the first belt 106 placed in a direct facing relationship with the wearer facing surface 117b of the second belt 108, the first surfaces 308 of the first flange 300a and the second flange 300b are in a direct facing relationship with the wearer facing surface 115b of the first belt 106. In addition, the second surfaces 310 of the first flange 300a and the second flange 300b are in a direct facing relationship with the wearer facing surface 117b of the second belt 108. As shown in FIG. 2A, when the diaper pant 100P is in an expanded state, such as when being worn or being placed on a wearer, with the wearer facing surface 115b of the first belt 106 separated from the wearer facing surface 117b of the second belt 108, the first surfaces 308 of the first flange 300a and the second flange 300b define wearer facing surfaces and may be in a direct facing relationship with a wearer. In addition, the second surfaces 310 of the first flange 300a and the second flange 300b define garment facing surfaces and are maintained in a direct facing relationship with the wearer facing surface 117a of the second belt 108.

As discussed herein and as illustrated in the accompanying figures, it is to be appreciated that the first belt 106 and/or second belt 108 may be configured as laminates that may comprise regions having different numbers of layers of substrates. As such, it is to be appreciated that flanges may be bonded with and refastenably connected with various arrangements of layers of substrates of the first belt 106 and/or the second belt 108.

For example, FIG. 2C1 is a cross sectional view of first and second belts 106, 108 configured with laminate structures that correspond with laminate structures shown and described above with reference to FIG. 4. As shown in FIG. 2C1, the first surface 308 of the flange 300 may be bonded with the wearer facing surface 115b of the first belt 106, and the second surface 310 of the flange 300 may be refastenably connected with the wearer facing surface 117b of the second belt 108. More particularly, the first surface 308 of the flange 300 may be bonded with the second substrate 164 of the first belt 106, and a fastener component 304 on the second surface 310 of the flange 300 may be refastenably connected with the second substrate 164 of the second belt 108.

In other configurations described above, at least one substrate of a first belt 106 and/or a second belt 108 may be folded to partially overlap itself and/or another substrate, which in turn may define regions of the belt having different numbers of layers of substrates. For example, FIG. 2C2 is a cross sectional view of first and second belts 106, 108 configured with laminate structures that correspond with laminate structures shown and described above with reference to FIGS. 4A, 4B, and 4A1. As shown in FIG. 2C2, the first surface 308 of the flange 300 may be bonded with the second substrate 164 as well as the folded portion 162g of the first substrate 162 of the first belt 106. In addition, a fastener component 304 on the second surface 310 of the flange 300 may be refastenably connected with the second substrate 164 as well as the folded portion 162g of the first substrate 162 of the second belt 108.

FIG. 2C3 shows yet another cross sectional view of first and second belts 106, 108 configured with laminate structures that correspond with laminate structures shown and described above with reference to FIG. 4A2. As shown in FIG. 2C3, the first surface 308 of the flange 300 may be bonded with the second substrate 164 in regions where the second substrate 164 overlaps the folded portion 162g of the first substrate 162 of the first belt 106. In addition, a fastener component 304 on the second surface 310 of the flange 300 may be refastenably connected with the second substrate 164 in regions where the second substrate 164 overlaps the folded portion 162g of the first substrate 162 of the second belt 108.

In other configurations, second belt 108 may comprise a stiffening patch 400. FIG. 2C4 shows a cross sectional view of first and second belts 106, 108 configured with laminate structures that correspond with laminate structures shown and described above with reference to FIGS. 4A, 4B, and 4A1, wherein second belt 108 comprises a stiffening patch 400. As shown in FIG. 2C4, the stiffening patch 400 may comprise a first surface 401 and a second surface 403, wherein the second surface 403 may be bonded with a portion of the second substrate 164 of the second belt 108 and a portion of the folded portion 162g of the first substrate 162 of the second belt 108. In addition, a fastener component 304 on the second surface 310 of the flange 300 may be refastenably connected with the first surface 401 of the stiffening patch 400.

FIG. 2C5 shows yet another cross sectional view of first and second belts 106, 108 configured with laminate structures that correspond with laminate structures shown and described above with reference to FIGS. 4A, 4B, and 4A1, wherein second belt 108 comprises a stiffening patch 400. As shown in FIG. 2C5, the stiffening patch 400 may be positioned between the first substrate 162 and the second substrate 164 of the second belt 108. In addition, a fastener component 304 on the second surface 310 of the flange 300 may be refastenably connected with the second substrate 164 as well as the folded portion 162g of the first substrate 162 of the second belt 108.

FIG. 2C6 shows yet another cross sectional view of first and second belts 106, 108 configured with laminate structures that correspond with laminate structures shown and described above with reference to FIGS. 4A, 4B, and 4A1, wherein second belt 108 comprises a stiffening patch 400. As shown in FIG. 2C6, the second surface 403 of the stiffening patch 400 may be bonded with the second substrate 164 of the second belt 108 and the folded portion 162g of the first substrate 162 of the second belt may overlap and be bonded with a portion of the first surface 401 of the stiffening patch 400.

Stiffening patches 400 may be connected with the belt in various ways, such as mechanical bonding, adhesive bonding, or both. While FIGS. 2C4-2C6 illustrate stiffening patch 400 positioned on second belt 108, it is to be appreciated that in some configurations where flanges 300 may be bonded with the opposing end regions of the second belt, the stiffening patches may be positioned on the first belt 106.

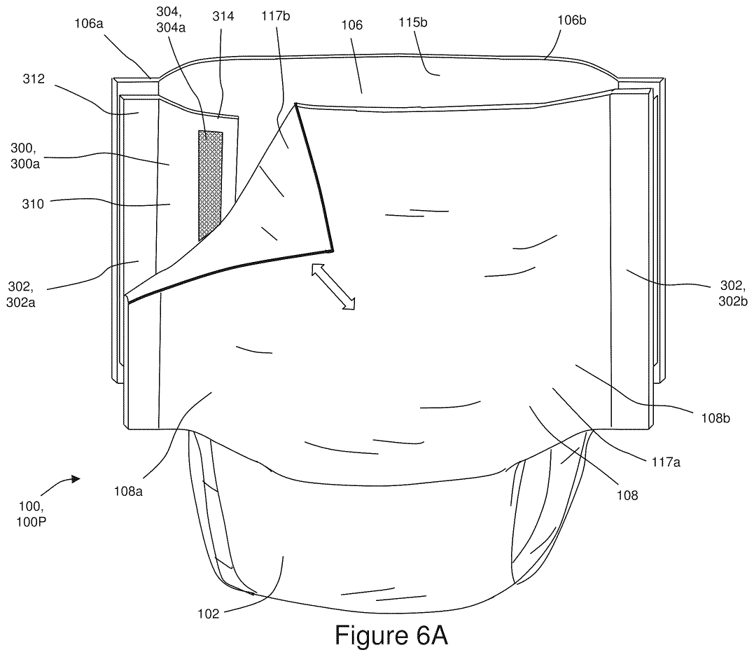

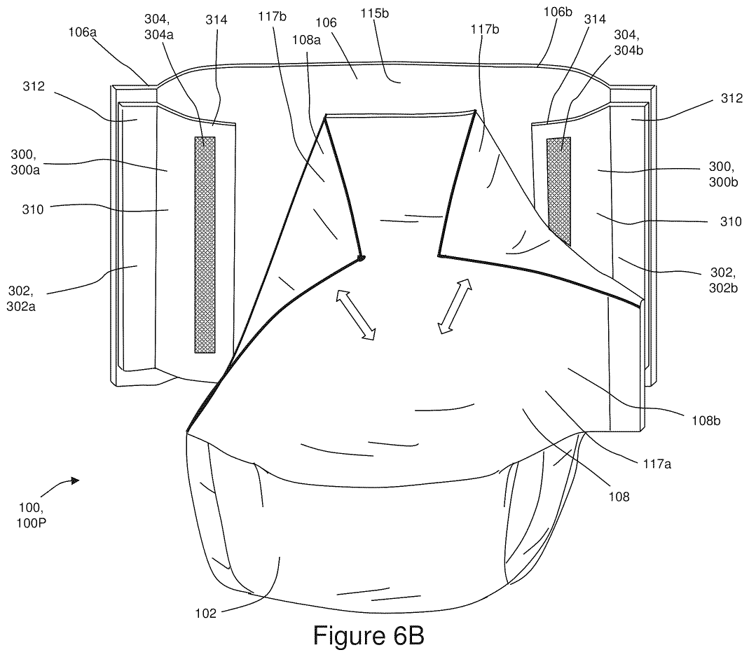

As discussed above, the diaper pants 100P described herein may include one or more refastenable connections between the first belt 106 and the second belt 108. For example, FIGS. 1, 6A, and 6B show an example diaper pant 100P with a first belt 106 refastenably connected with the second belt 108. The refastenable connections between the belts may allow the first elastic belt 106 and the second belt 108 to be partially or completely separated from each other, such as when inspecting a diaper pant 100P while being worn in order to determine if the diaper pant 100P may be soiled. If it is determined that the diaper pant 100P is suitable for continued use, the first and second belts 106, 108 may be reconnected with each other. For example, FIG. 6A is a perspective view of the diaper pant 100P of FIG. 1 showing the first end region 108a of the second belt 108 partially disconnected from the first flange 300a. From the configuration shown in FIG. 6A, it is to be appreciated that the disconnected portion of the first end region 108a of the second belt 108 may be reconnected with the first fastener component 304a with the application of forces to the second belt 108 generically represented by a bi-directional arrow. It is also to be appreciated that the remaining connected portion of the first end region 108a of the second belt 108 may be further disconnected from the first fastener component 304a with the application of forces to the second belt 108 generically represented by a bi-directional arrow.

FIG. 6B is a perspective view of the diaper pant of FIG. 6A showing the first end region 108a of the second belt 108 having been completely disconnected from the first fastener component 304a on the first flange 300a. FIG. 6B also shows the second end region 108b of the second belt 108 partially disconnected from a second flange 300b. From the configuration shown in FIG. 6B, it is to be appreciated that the disconnected portions of the first end region 108a and the second end region 108b of the second belt 108 may be reconnected with the first and second fastener components 304a, 304b, respectively, with the application of forces to the second belt 108 generically represented by the bi-directional arrows. It is also to be appreciated that the remaining connected portion of the second end region 108b of the second belt 108 may be further disconnected from the second fastener component 304b with the application of forces to the second belt 108 generically represented by a bi-directional arrow.

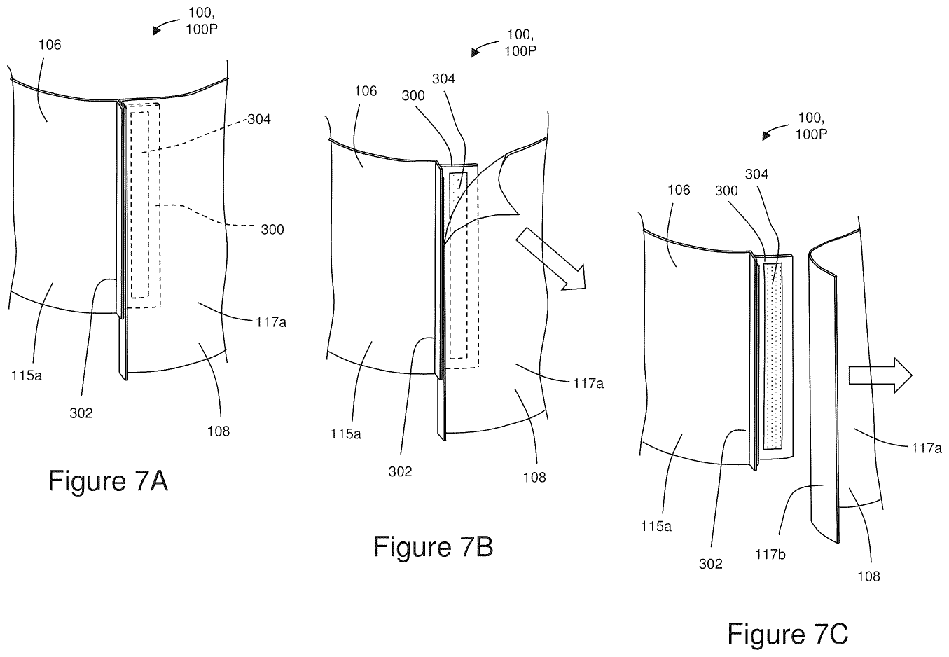

As previously discussed, the refastenable connections between the belts 106, 108 may be configured to allow the first elastic belt 106 and the second elastic belt 108 to be relatively easily and completely separated from each other, such as when removing the diaper pant 100P from a wearer. For example, FIGS. 7A-7C illustrate a progression whereby the first belt 106 and the second belt 108 may be completely disconnected from each other. In particular, FIG. 7A is a detailed view of a diaper pant 100P showing a flange 300 bonded with a first belt 106 and refastenably connected with a second belt 108. FIG. 7B is a detailed view of the diaper pant 100P of FIG. 7A showing the second belt 108 partially disconnected from the flange 300, and FIG. 7C is a detailed view of the diaper pant 100P of FIG. 7B showing the second belt 108 completely disconnected from the flange 300. As such, once the opposing end portions of the first elastic belt 106 and the second elastic belt 108 are completely disconnected, the diaper pant 100P may be removed from a wearer in a similar fashion to a taped diaper.

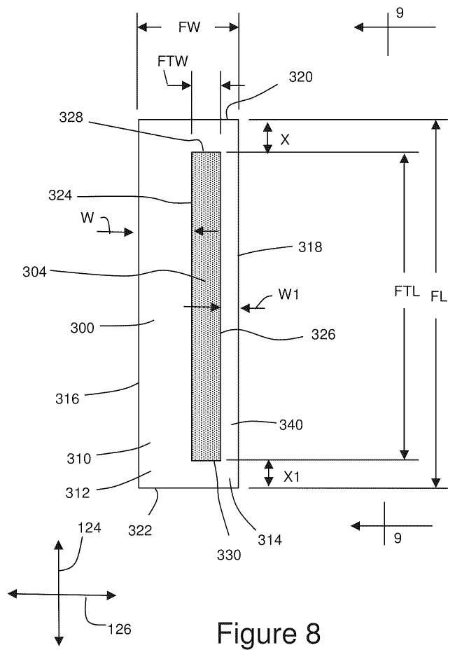

It is to be appreciated that the flange 300 and/or the fastener component 304 may comprise any of a wide variety of shapes, including rectangles or other polygons, circles, ovals, shapes having exterior convexities or concavities or combinations thereof, or one or a plurality of lines or geometric shapes forming an array. It is also to be appreciated that more than one fastener component 304 may be positioned on a flange. It is also to be appreciated that the fastener component 304 and the flange 300 may be configured with various sizes and shapes. For example, FIG. 8 illustrates a detailed planar view of a fastener component 304 positioned on the second surface 310 of a flange 300. To provide a frame of reference, a longitudinal axis 124 and a lateral axis 126 are illustrated in FIG. 8 and correspond with the directional frames of reference provided by the longitudinal axis 124 and lateral axis 126, respectively, shown in other figures herein. As shown in FIG. 8, the flange 300 may define a lateral width FW and longitudinal length FL, and the fastener component 304 may define a lateral width FTW and a longitudinal length FTL. In some configurations, FW may be equal to or greater than FTW, and FL may be equal to or greater than FTL. The flange 300 may have a lateral width FW of from about 26 mm to about 40 mm, or from about 30 mm to about 38 mm, and a longitudinal length FL of from about 110 mm to about 130 mm, or from about 115 mm to about 125 mm. The fastener component 304 may have a lateral width FTW of from about 10 mm to about 18 mm, or from about 12 mm to about 15 mm, and a longitudinal length FTL of from about 105 mm to about 120 mm, or from about 108 mm to about 115 mm. In some configurations, gap regions 340 may be defined on the flange 300 by the absence of portions of the fastener component 304.

With continued reference to FIG. 8, the flange 300 may comprise a first side edge 316 laterally separated from a second side edge 318 and may comprise a first end edge 320 longitudinally separated from a second end edge 322. In addition, the fastener component 304 may comprise a first side edge 324 laterally separated from a second side edge 326 and may comprise a first end edge 328 longitudinally separated from a second end edge 330. In some configurations, the first side edge 324 of fastener component 304 may be coterminous with or laterally inboard from the first side edge 316 of the flange 300, and/or the second side edge 326 of the fastener component 304 may be coterminous with or laterally inboard from the second side edge 318 of the flange 300. In some configurations, the first side edge 324 of fastener component 304 may be positioned laterally inboard from the first side edge 316 of flange 300 a distance W of from about 8 mm to about 21 mm, or from about 10 mm to about 18 mm, and/or the second side edge 326 of fastener component 304 may be positioned laterally inboard from the second side edge 318 of flange 300 by a distance W1 of from about 1 mm to about 9 mm, or from about 3 mm to about 7 mm. Without being limited by theory, it is believed that if the fastener component 304 is positioned greater than about 9 mm from the second side edge 318 of flange 300, gap region 340 of flange 300 may fold over and cover a portion of fastener component 304, thus interfering with the engagement of fastener component 304 with the opposing belt.

In some configurations, the first end edge 328 of the fastener component 304 may be coterminous with or longitudinally inboard from first end edge 320 of the flange 300, and/or the second end edge 330 of the fastener component 304 may be coterminous with or longitudinally inboard from second end edge 322 of the flange 300. In some configurations, the first end edge 328 of fastener component 304 may be positioned longitudinally inboard from first end edge 320 of flange 300 by a distance X of from about 2 mm to about 8 mm, or from about 4 mm to about 6 mm, and/or the second end edge 330 of fastener component 304 may be positioned longitudinally inboard from second end edge 322 of flange 300 by a distance X1 of from about 2 mm to about 8 mm, or from about 4 mm to about 6 mm. Without being limited by theory, it is believed that such placement of the first end edge 328 and/or second end edge 330 of fastener component 304 with respect to the first and/or second end edges 320, 330 of flange 300 may help to avoid the fastener component 304 from protruding longitudinally outboard of the first waist edge and coming into contact with the skin during use.

It is also to be appreciated that the fastener component may be configured in various ways and may be connected with the flange in various ways. As discussed above, the fastener component may comprise a hook material that can refastenably engage with substrates, such as nonwovens for example. For example, the fastener component may comprise a base or laminate structure comprising protrusions, such as hooks, wherein the base or laminate structure is bonded with the flange (or a belt), which may comprise a nonwoven. It is to be appreciated that the base or laminate structure may be bonded with the flange (or a belt) in various ways, such as for example, with mechanical bonds, thermal bonds, ultrasonic bonds, and/or adhesive bonds or combinations thereof.

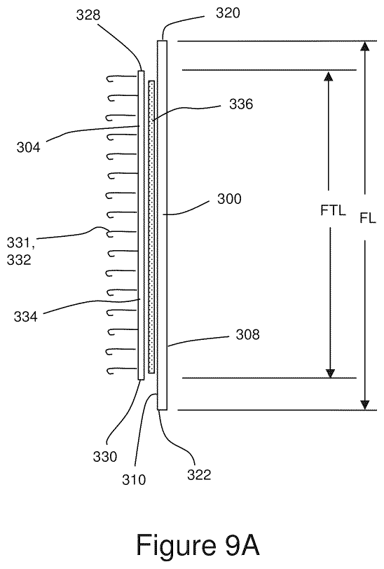

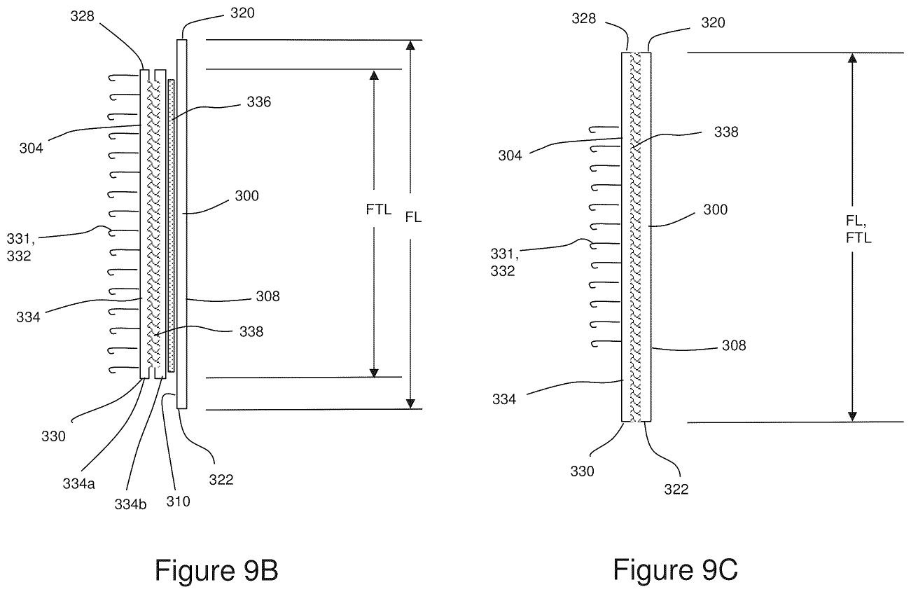

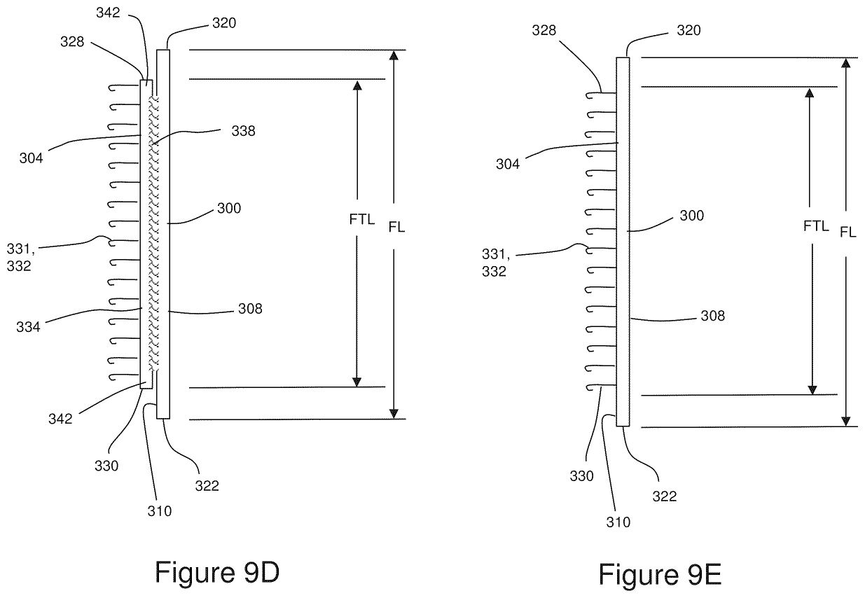

For example, as shown in FIG. 9A, the fastener component 304 may comprise a plurality of protrusions 331 extending from a base 334. Protrusions 331 may be hooks, nubs, or other protrusions having various shapes configured to engage a receiving component or another fastener component. In some configurations, the fastener component 304 may comprise hooks 332 protruding from a base 334, and adhesive 336 may connect the base 334 of the fastener component 304 with the second surface 310 of the flange 300. It is to be appreciated that the fastener component 304 may be connected with flange 300 by mechanical bonding in addition to or instead of adhesive 336. It is also to be appreciated that the base 334 may be configured in various ways. For example, the base 334 may comprise a thermoplastic film. In addition, the adhesive 336 between the base 334 and the flange 300 may extend longitudinally for the entire length FTL or less than the entire length FTL of the base. Further, the adhesive 336 between the base 334 and the flange 300 may extend laterally for the entire width FTW or less than the entire length FTW of the base.

In some configurations, the base 334 may comprise a laminate with various layers bonded together. It is also to be appreciated that such layers may be bonded together in various ways, such as with adhesive, mechanical bonding, and/or extrusion bonding. In some configurations, layers of the base 334 may be bonded together with extrusion or melt type bonding such as disclosed for example in U.S. Patent Publication No. 2021/0045931A1. For example, as shown in FIG. 9B, the base 334 may comprise a thermoplastic film layer 334a and a nonwoven backing layer 334b, wherein the thermoplastic film layer 334a is bonded with the nonwoven backing layer 334b with extrusion bonds 338. In turn, the nonwoven backing layer 334b may be bonded with the flange 300 with adhesive 336.

In some configurations, such as shown in FIGS. 9C and 9D for example, the base may be bonded directly with the flange with extrusion or melt type bonding, such as disclosed for example in U.S. Patent Publication No. 2021/0045931A1. For example, the base 334 may comprise a thermoplastic film layer and the flange 300 may comprise a nonwoven layer, wherein the thermoplastic film base 334 is bonded with the nonwoven flange 300 with extrusion bonds 338. In some configurations, such as shown in FIG. 9C, the base 334 may extend longitudinally for the entire length FL of the flange 300, such as from the first end edge 320 of the flange 300 to the second end edge 322 of the flange 300, and the hooks 332 may extend longitudinally for less than the entire length FL of the flange 300. In addition, the extrusion bonds 338 between the base 334 and the flange 300 may extend longitudinally for the entire lengths FL, FTL of the flange 300 and base 334. Further, the extrusion bonds 338 between the base 334 and the flange 300 may extend laterally for the entire width FTW or less than the entire length FTW of the base 334. In some configurations, such as shown in FIG. 9D, the base 334 may extend longitudinally for a length FTL that is less than the entire length FL of the flange 300. In addition, the extrusion bonds 338 between the base 334 and the flange 300 may extend longitudinally for the entire length FTL or less than the entire length FTL of the base 334. Further, the extrusion bonds 338 between the base 334 and the flange 300 may extend laterally for the entire width FTW or less than the entire length FTW of the base 334. As such, in some configurations, edge regions 342 of the base 334 adjacent the first side edge 324, the second side edge 326, the first end edge 328, and/or the second end edge 330 may not be bonded with the flange 300.

In some configurations, protrusions 331 may be integrally formed from the flange 300. In some configurations, protrusions 331 may be hooks 332 integrally formed from the flange 300, which may for example be in the form of a nonwoven. For example, as shown in FIG. 9E, the fastener component 304 may be integrally formed from materials of the flange 300 or may be integrally formed from other materials that may then be attached with the flange 300.

It is to be appreciated that the discussions and descriptions above with regard to bonding arrangements and configurations between the fastener components 304 and the flange 300 are also applicable to bonding arrangements and configurations between the fastener components 304 and materials of first and/or second belts 106, 108.

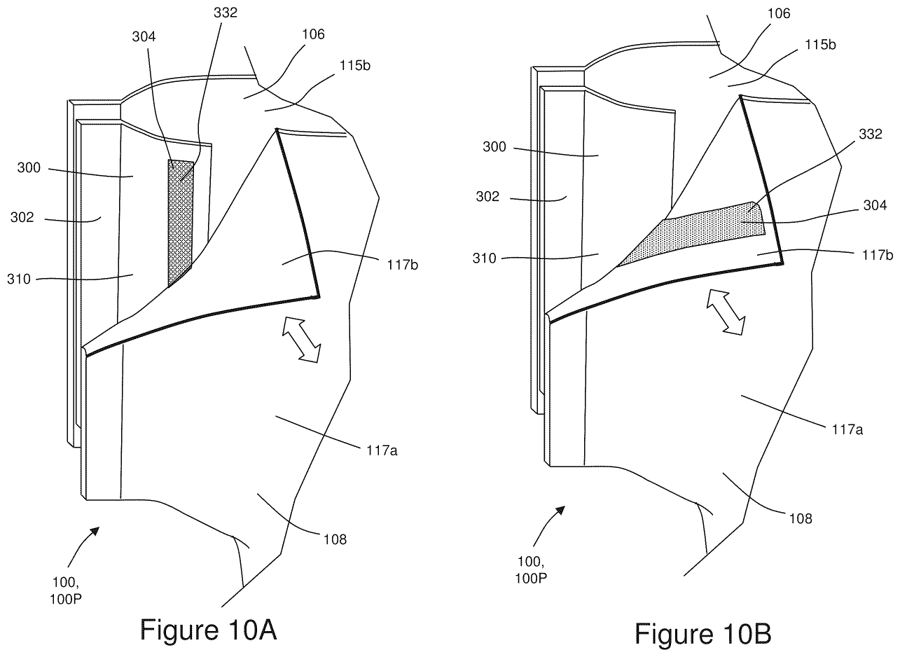

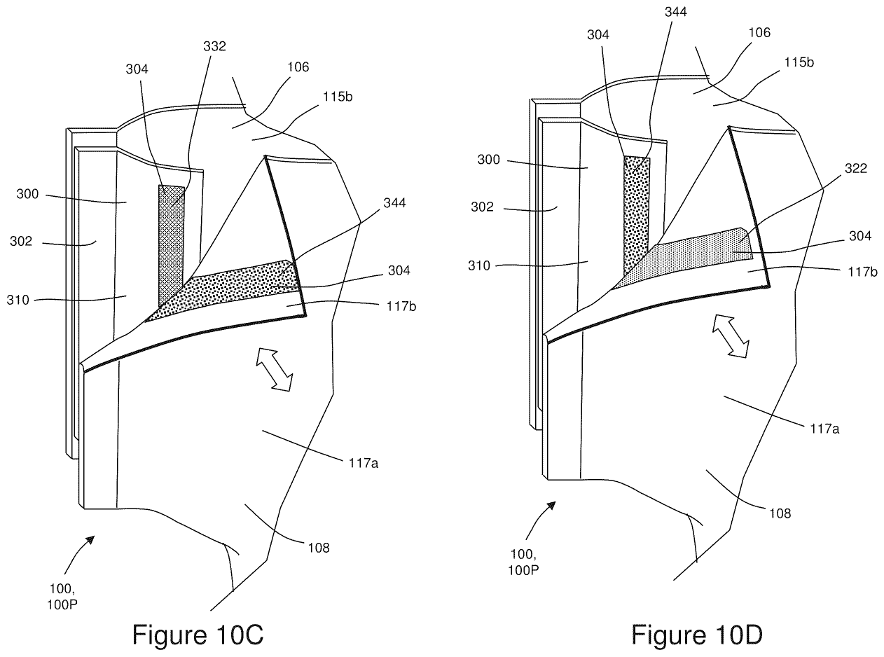

As discussed above, it is to be appreciated that the various arrangements and types of fastener components 304 and flanges 300 may be configured to refastenably connect opposing end regions of the first belt 106 with the second belt 108. For example, FIG. 10A is a detailed view of a diaper pant 100P showing a second belt 108 directly refastenably connected with a fastener component 304 on a flange 300. The fastener component 304 may comprise hooks 332 adapted to directly refastenably connect with material defining the wearer facing surface 117b of the second belt 108, such as for example the first substrate 162 and/or 164 of the second belt 108. In another example, FIG. 10B shows a detailed view of a diaper pant 100P with a fastener component 304 comprising hooks 332 on a wearer facing surface 117b of the second belt 108 that may be adapted to directly refastenably connect with material defining the second surface 310 of the flange 300. In yet another example, FIG. 10C shows a detailed view of a diaper pant 100P with a fastener component 304 comprising hooks 332 on the second surface 310 of the flange 300 and a fastener component 304 comprising loops 344 on the wearer facing surface 117b of the second belt 108, wherein the hooks 332 are adapted to refastenably connect with the loops 344. In still another example, FIG. 10D a detailed view of a diaper pant 100P with a fastener component 304 comprising loops 344 on the second surface 310 of the flange 300 and a fastener component 304 comprising hooks 332 on the wearer facing surface 117b of the second belt 108, wherein the hooks 332 are adapted to refastenably connect with the loops 344. It is to be appreciated that in some configurations, adhesive may be used in place of or in addition to the hooks as described above with reference to FIGS. 10A-10D.

As discussed above, first belt 106 and/or the second belt 108 may be assembled in various ways and various combinations to create various desirable features that may differ along the lateral width and/or longitudinal length of the first belt 106 and/or the second belt 108. In some configurations, first belt 106 and/or second belt 108 may comprise one or more elastic zones extending in the lateral direction. In some configurations, the first belt 106 and/or the second belt 108 may comprise two elastic zone, or three elastic zones, or four or more elastic zones. Elastic zones may be continuous or discontinuous, as for example, when disrupted by chassis 102.

In some configurations, the tensile force (N) of the first and second belts 106, 108 may be profiled such that the tensile force of each elastic zone may vary along the longitudinal length of the belt. In some configurations, the first belt may have a profiled tensile force and the second belt may have a substantially uniform tensile force.

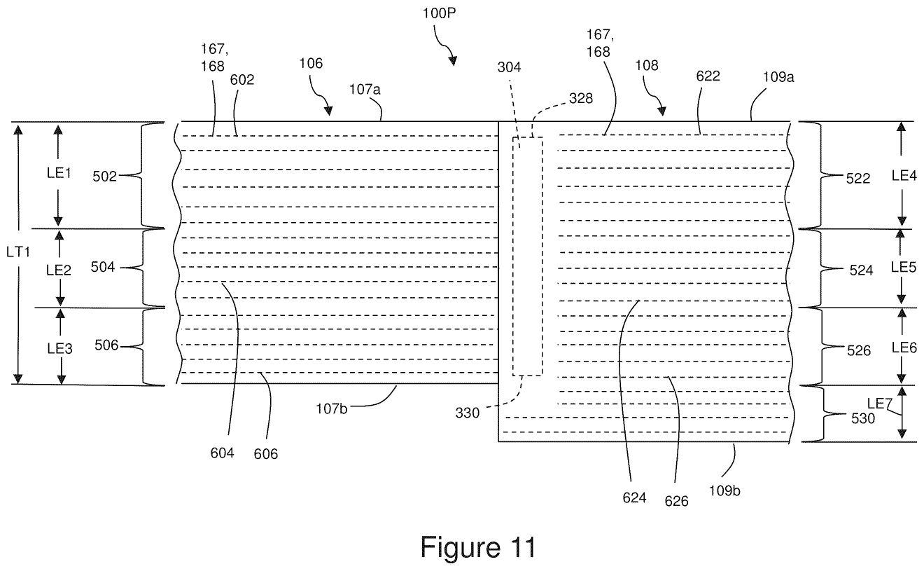

FIG. 11 is a side plan view of diaper pant 100P in a flat uncontracted condition showing the garment facing surface, illustrating example elastic material 167 arrangements in the first and second belts 106, 108. As shown in FIG. 11, first belt 106 may comprise a first upper elastic zone 502 positioned adjacent to the outer laterally extending edge 107a, a first lower elastic zone 506 positioned adjacent to the inner laterally extending edge 107b, and a first central elastic zone 504 disposed therebetween. The first upper elastic zone 502 may comprise a first longitudinal length LEI that is about 40% of the first belt longitudinal length LT1. The first central elastic zone 504 may comprise a second longitudinal length LE2 that is about 30% of the first belt longitudinal length LT1. The first lower elastic zone 506 may comprises a third longitudinal length LE3 that is about 30% of the first belt longitudinal length LT1.

In some configurations, the second belt 108 may comprise a second upper elastic zone 522 positioned adjacent to the outer laterally extending edge 109a, a second central elastic zone 524 positioned longitudinally inboard of the second upper elastic zone 522, and a second lower elastic zone 526 positioned longitudinally inboard of the second central elastic zone 524. In some configurations, the second belt 108 may further comprise a second lower leg elastic zone 530 positioned adjacent to the inner laterally extending edge 109b. The second upper elastic zone 522 may comprise a fourth longitudinal length LE4 that is about 40% of the first belt longitudinal length LT1. The second central elastic zone 524 may comprise a fifth longitudinal length LE5 that is about 30% of the first belt longitudinal length LT1. The second lower elastic zone 526 may comprise a sixth longitudinal length LE6 that is about 30% of the first belt longitudinal length LT1. The second lower leg elastic zone 530 may comprise a seventh longitudinal length LE7.

Each elastic zone may comprise an elastic material 167. In some configurations, each elastic zone may comprise a plurality of elastic strands 168. It is to be appreciated that each elastic zone is separated by cutting through on the longitudinal center between the bottom elastic strand of one zone and the top elastic strand of the adjacent zone, with care not to cut any portion of the elastic strands substantially in parallel with each other in the lateral direction. If any of the elastic strands is on the border of an elastic zone, the zone is separated at the longitudinal center between the elastic on the border and the adjacent elastic closest to the central elastic zone.

Elastic strands 168 may be distributed evenly amongst the elastic zones. Elastic strands 168 may also be distributed unevenly amongst the different elastic zones. Elastic strands 168 located in the first and second central elastic zones 504, 524 and the first and second lower elastic zones 506, 526 may be laterally interrupted by the chassis 102. The first upper elastic zone 502 may comprise a first plurality of elastic strands 602 and has a first tensile force. The first central elastic zone 504 may comprise a second plurality of elastic strands 604 and has a second tensile force. The first lower elastic zone 506 may comprise a third plurality of elastic strands 606 and has a third tensile force. The second upper elastic zone 522 may comprise a fourth plurality of elastic strands 622 and has a fourth tensile force. The second central elastic zone 524 may comprise a fifth plurality of elastic strands 624 and has a fifth tensile force. The second lower elastic zone 526 may comprise a sixth plurality of elastic strands 626 and has a sixth tensile force. The second lower leg elastic zone 530 may comprise a seventh plurality of elastic strands 630 and has a seventh tensile force.

In some configurations, the second tensile force may be less than or equal to the third tensile force. In some configurations, the second tensile force and/or the third tensile force may be less than about 1.20 N. In some configurations, the second tensile force and/or the third tensile force may be from about 0.10 N to about 1.20 N, or from about 0.50 N to about 1.10 N. In some configurations, the second tensile force may be from about 5% to about 80% less than the third tensile force. In some configurations, the second and third tensile forces may be less than about 2.00 N. In some configurations, the fifth tensile force may be less than or equal to the sixth tensile force. The fifth tensile force and/or the sixth sensile force may be from about 0.50 N to about 2.30 N, or from about 0.80 N to about 2.10 N. The fifth tensile force may be from about 0% to about 30% less than the sixth tensile force. In some configurations, the fifth and sixth tensile forces may be less than about 3.00 N. In some configurations, the first and fourth tensile forces may be from about 0.50 N to about 3.00 N. Without being limited by theory, it is believed that by having the second and/or fifth tensile forces less than the third and/or sixth tensile forces, respectively, the elastic belts are more easily fastened around the wearer's waist yet still provides good fit and comfort to the wearer. It is believed that when a caregiver places the refastenable absorbent article around the waist of a wearer, the caregiver will first secure the first end edge area of the fastener component to the opposing belt. If the second and/or fifth tensile force is greater than the third and/or sixth tensile force, respectively, the fastener component can buckle and/or bend, causing a large gap and making it difficult for the caregiver to secure the full length of the fastener component to the opposing belt.

In some configurations, the diaper pant 100P may have a Fit Circumference Force of greater than 3 N, or from about 3 N to about 6 N, or from about 4 N to about 5 N, according to the Whole Article Force Measurement as described herein below.

In some configurations the first, second, third, fourth, fifth, and/or sixth plurality of elastic strands may have an Average Dtex of from about 10 to about 1000. In some configurations, the first and/or fourth plurality of elastic strands 602, 622 may have an Average Dtex of from about 200 to about 800, or from about 300 to about 700. The second and/or fifth plurality of elastic strands 604, 624 may have an Average Dtex of from about 200 to about 800, or from about 300 to about 700. The third and/or sixth plurality of elastic strands 606, 626 may have an Average Dtex of from about 400 to about 1000.

In some configurations the first, second, third, fourth, fifth, and/or sixth plurality of elastic strands may have an Average Pre-Strain of from about 150% to about 300%. In some configurations, the first and/or fourth plurality of elastic strands 602, 622 may have an Average Pre-Strain of less than about 300%, or from about 150% to about 300%, or from about 180% to about 300%, or from about 220% to about 300%. The second and/or fifth plurality of elastic strands 604, 624 may have an Average Pre-Strain of less than about 300%, or from about 150% to about 275%, or from about 180% to about 275%. The third and/or sixth plurality of elastic strands 606, 626 may have an Average Pre-Strain of less than about 300%, or from about 250% to about 275%. In some configurations, the Average Pre-Strain of the second and/or fifth plurality of elastic strands 604, 624 may be less than or equal to the Average Pre-Strain of the third and sixth plurality of elastic strands 606, 626.

In some configurations, the pitch (measured from the center of one elastic strand to the center of the adjacent elastic strand) between the elastic strands in the first upper elastic zone 502 and the first central elastic zone 504 may be larger than the pitch between the elastic strands in the first lower elastic zone 506. The pitch between the elastic strands in the first upper elastic zone 502 and the central elastic zone 504 may be from about 4 mm to about 16 mm, or from about 6 mm to about 12 mm, or from about 7 mm to about 9 mm. The pitch between the elastic strands in the first lower elastic zone 506 may be from about 4 mm to about 8 mm, or from about 5 mm to about 7 mm. The pitch between the elastic strands in the second upper, central, and lower elastic zones 522, 524, and 526 may be substantially equal. The gap between the elastic strands in the second upper, central, and lower elastic zones 522, 524, and 526 may from about 4 mm to about 16 mm, or from about 6 mm to about 12 mm, or from about 7 mm to about 9 mm. In some configurations, the pitch between the elastic strands of the first and/or second elastic belts 106, 108 may be from about 0.5 to about 10 mm.

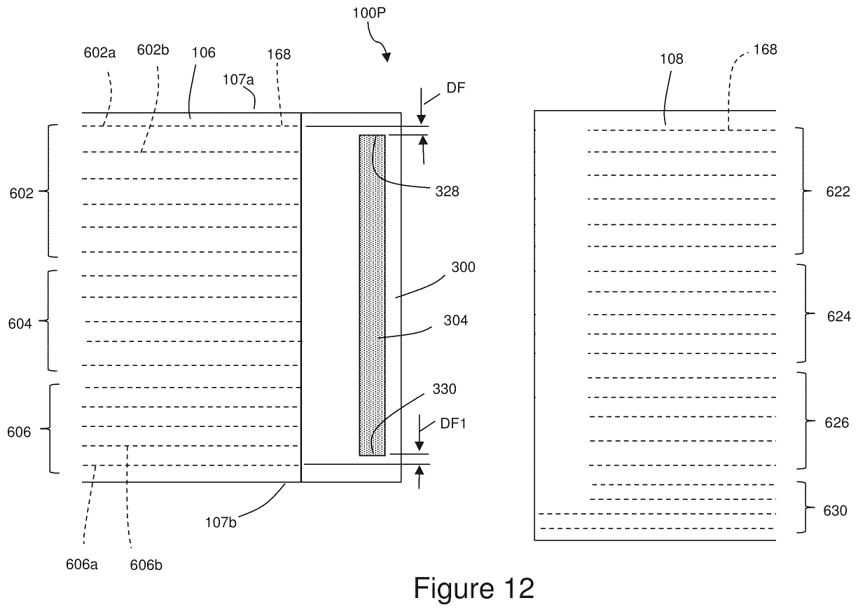

FIG. 12 is a side plan view of diaper pant 100P in a flat uncontracted state showing first belt 106 with fastener component 304 positioned on flange 300 prior to engagement with second belt 108, illustrating the positioning of fastener component 304 in relation to the elastic strands 168. As shown in FIG. 12, the first plurality of elastic strands 602 may comprise at least a first elastic strand 602a positioned adjacent to outer laterally extending edge 107a of first belt 106 and a second elastic strand 602b positioned longitudinally inboard of the first elastic strand 602a. In some configurations, the first end edge 328 of fastener component 304 may be positioned between the first elastic strand 602a and the second elastic strand 602b of the first plurality of elastic strands 602. In some configurations, the first end edge 328 of fastener component 304 may be positioned longitudinally outboard of the first elastic strand 602a of the first plurality of elastic strands 602. In some configurations, no more than one elastic strand may be positioned longitudinally outboard of the first end edge 328 of fastener component 304. In some configurations, the first elastic strand 602a and the first end edge 328 of fastener component 304 may be spaced apart from each other by a distance DF of from about 0 mm to about 8 mm, or from about 1 mm to about 6 mm, or from about 2 mm to about 4 mm. In some configurations, the third plurality of elastic strands 606 may comprise at least a first elastic strand 606a positioned adjacent to inner laterally extending edge 107b and a second elastic strand 606b positioned longitudinally inboard of the first elastic strand 606a. In some configurations, the first elastic strand 606a of the third plurality of elastic strands 606 and the second end edge 330 of fastener component 304 may be spaced apart from each other by a distance DF1 of from about 0 mm to about 8 mm, or from about 1 mm to about 6 mm, or from about 2 mm to about 4 mm.

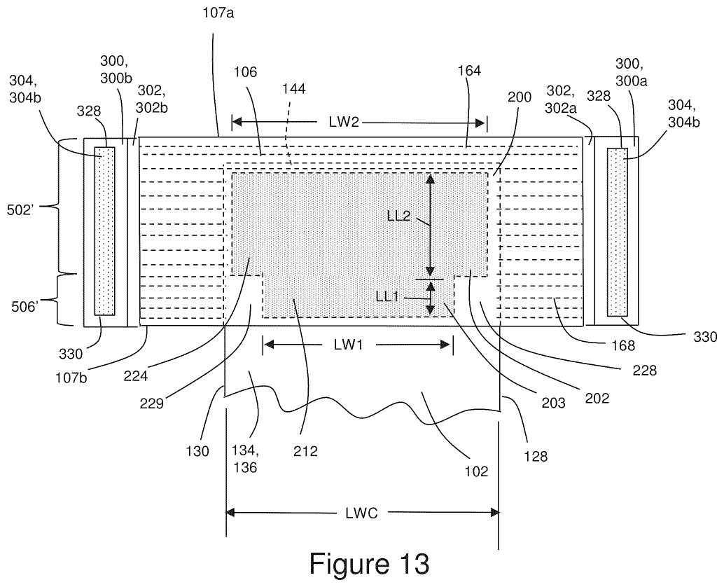

It is to be appreciated that various types of arrangements of adhesive may be used to connect the chassis 102 with the first belt 106 and the second belt 108. As shown for example in FIG. 13, a portion of the chassis 102 overlaps the wearer facing surface of the first belt 106 to define a chassis overlap region 200. The chassis overlap region 200 may extend laterally between the first longitudinal edge 128 and the second longitudinal edge 130 of the chassis 102 and longitudinally between the first lateral edge 144 of the chassis 102 and the inner laterally extending edge 107b of the first belt 106. The chassis overlap region 200 may comprise an attachment area 202 where adhesive 203 is positioned between the backsheet 136 of the chassis 102 and the wearer facing surface of the first belt 106. As such, the adhesive 203 in the attachment area 202 may bond the chassis 102 with the first belt 106. It is to be appreciated that the adhesive 203 may be applied in the attachment area 202 in various ways. For example, the attachment area 202 may comprise laterally extending stripes of adhesive 203 longitudinally separated from each other. In some configurations, the attachment area 202 may comprise a contiguous layer of adhesive 203. In some configurations, the attachment area 202 may comprise longitudinally extending stripes of adhesive laterally separated from each other.

The attachment area 202 may comprise a first attachment zone 212 and a second attachment zone 224. The first attachment zone 212 may be positioned adjacent the inner laterally extending edge 107b of the first belt 106, and the second attachment zone 224 may be positioned adjacent the first lateral edge 144 of the chassis 102. In some configurations, the first attachment zone 212 may be coterminous with or spaced longitudinally outboard from the inner laterally extending edge 107b of the first belt 106. In some configurations, the second attachment zone 224 may be coterminous with or spaced longitudinally inboard from the first end edge 144 of the chassis 102. In some configurations, the first attachment zone 212 and/or the second attachment zone 224 may be coterminous with or spaced laterally inboard from the first longitudinal edge 128 and/or the second longitudinal edge 130 of the chassis 102.

It is to be appreciated that the first attachment zone 212 and the second attachment zone 224 may comprise various relative longitudinal lengths and lateral widths. For example, the first attachment zone 212 may comprise a first lateral width LW1 and a first longitudinal length LL1 and the second attachment zone 224 may comprise a second lateral width LW2 and a second longitudinal length LL2. In some configurations, the first lateral width LW1 may be less than the second lateral width LW2. The first lateral width LW1 may be from about 10 to about 15% less than the second lateral width LW2. In some configurations, the first lateral width LW1 may be from about 150 mm to about 175 mm, or from about 155 mm to about 170 mm, and/or the second lateral width LW2 may be from about 180 mm to about 200 mm, or from about 185 mm to about 190 mm. The first lateral width LW1 may be from about 75% to about 85% of the lateral width LWC of the chassis overlap region 200. In some configurations, the first longitudinal length LL1 may be less than the second longitudinal length LL2. The first longitudinal length LL1 may be from about 30 to about 40% of the second longitudinal length LL2. In some configurations, the first longitudinal length LL1 may be from about 25 mm to about 40 mm, or from about 30 mm to about 35 mm and/or the second longitudinal length LL2 may be from about 70 mm to about 85 mm, or from about 75 mm to about 80 mm.

With continued reference to FIG. 13, the attachment region 202 may be configured to define a first non-bonded region 228 and a second non-bonded region 229 in the chassis overlap region 200. The chassis 102 may not be bonded with the first belt 106 in the first and second non-bonded regions 228, 229. The first non-bonded region 228 may extend laterally between the first attachment zone 212 and the first longitudinal side edge 128 of the chassis 102, and the second non-bonded region 229 may extend laterally between the first attachment zone 212 and the second longitudinal side edge 130 of the chassis 102. In some configurations, the first non-bonded region 228 and/or the second non-bonded region 229 may have a longitudinal length of from about 10 mm to about 60 mm, or from about 20 mm to about 50 mm. It was found that because chassis 102 and the first belt 106 are not bonded together in the non-bonded regions 228, 229, the problem of inward belt curling of the first belt during use (and the resulting hook-to-skin contact on the thigh) may be reduced and thus may provide a more comfortable fit. It is to be appreciated that the description above with regard to the first belt and chassis bonding arrangements and configurations may also be applicable to bonding arrangements and configurations between the second belt and the chassis.

In some configurations, as shown in FIG. 13, first belt 106 may comprise a laterally extending first upper elastic zone 502′ and a laterally extending first lower elastic zone 506′. First lower elastic zone 506′ may be positioned longitudinally inboard of the second attachment zone 224 and laterally outboard of the first attachment zone 212. First upper elastic zone 502′ may be positioned longitudinally outboard of the first lower elastic zone 506′. In some configurations, the first lower elastic zone 506′ may have a tensile force of less than about 1.20 N. In some configurations, the first lower elastic zone 506′ may have a tensile force of from about 0.10 N to about 1.20 N, or from about 0.50 N to about 1.10 N. In some configurations, the first upper elastic zone 502′ may comprise more than one zone, such a first upper elastic zone and a first central elastic zone as previously described.

In some configurations, the second end edge 330 of fastener component 304 may be positioned longitudinally inboard of the second attachment zone 224 and laterally outboard of the first attachment zone 212. In some configurations, the first lower elastic zone 506, 506′ may be adjacent to the second end edge 330 of fastener component 304.

The first belt 106, second belt 108, and/or flange 300 may comprise one or more design elements adapted to indicate the proper alignment of the first belt 106, second belt 108, and/or flange 300 to create a ring-like elastic belt 104 including one or more of insignia, letters, words, graphics, logos, colors, fonts, shapes, or combinations thereof. In some configurations, the first and second belts 106, 108 may have a first color and the flange 300 may comprise a second color that is different from the first color. When the first belt and second belts 106, 108 are connected, the second color of the flange 300 may be substantially covered by the belts, signaling to the caregiver that the belts are properly connected and provide confidence that the absorbent article is properly fitted to the user. In other configurations, the first belt 106 may comprise a first portion of a graphic and the second belt may comprise a second portion of the graphic When the first and second belts are connected, the graphic may be completed, signaling to the caregiver that the belts are properly connected and provide confidence that the absorbent article is properly fitted to the user.

The one or more design elements may be printed on the garment facing surface of the first belt 106, second belt 108, and/or flanges 300. The design elements may extend substantially around the entire circumference of the diaper pant 100P and may be disposed across longitudinal side edges of the first and second belts 106, 108 in the manner described in U.S. Pat. No. 9,498,389 to create a more underwear-like article.

### Average Decitex (Average-Dtex)

The Average Decitex Method is used to calculate the Average-Dtex on a length-weighted basis for elastic fibers present in an entire article, or in a specimen of interest extracted from an article. The decitex value is the mass in grams of a fiber present in 10,000 meters of that material in the relaxed state. The decitex value of elastic fibers or elastic laminates containing elastic fibers is often reported by manufacturers as part of a specification for an elastic fiber or an elastic laminate including elastic fibers. The Average-Dtex is to be calculated from these specifications if available. Alternatively, if these specified values are not known, the decitex value of an individual elastic fiber is measured by determining the cross-sectional area of a fiber in a relaxed state via a suitable microscopy technique such as scanning electron microscopy (SEM), determining the composition of the fiber via Fourier Transform Infrared (FT-IR) spectroscopy, and then using a literature value for density of the composition to calculate the mass in grams of the fiber present in 10,000 meters of the fiber. The manufacturer-provided or experimentally measured decitex values for the individual elastic fibers removed from an entire article, or specimen extracted from an article, are used in the expression below in which the length-weighted average of decitex value among elastic fibers present is determined.

The lengths of elastic fibers present in an article or specimen extracted from an article is calculated from overall dimensions of and the elastic fiber pre-strain ratio associated with components of the article with these or the specimen, respectively, if known. Alternatively, dimensions and/or elastic fiber pre-strain ratios are not known, an absorbent article or specimen extracted from an absorbent article is disassembled and all elastic fibers are removed. This disassembly can be done, for example, with gentle heating to soften adhesives, with a cryogenic spray (e.g., Quick-Freeze, Miller-Stephenson Company, Danbury, CT), or with an appropriate solvent that will remove adhesive but not swell, alter, or destroy elastic fibers. The length of each elastic fiber in its relaxed state is measured and recorded in millimeters (mm) to the nearest mm.

### Calculation of Average-Dtex

For each of the individual elastic fibers fᵢ of relaxed length Lᵢ and fiber decitex value dᵢ (obtained either from the manufacturer's specifications or measured experimentally) present in an absorbent article, or specimen extracted from an absorbent article, the Average-Dtex for that absorbent article or specimen extracted from an absorbent article is defined as:

$$Average - Dtex = \frac{{\sum }_{i = 1}^{n} ( {L}_{i} \times {d}_{i} )}{{\sum }_{i = 1}^{n} {L}_{i}}$$

where n is the total number of elastic fibers present in an absorbent article or specimen extracted from an absorbent article. The Average-Dtex is reported to the nearest integer value of decitex (grams per 10 000 m).

If the decitex value of any individual fiber is not known from specifications, it is experimentally determined as described below, and the resulting fiber decitex value(s) are used in the above equation to determine Average-Dtex.

### Experimental Determination of Decitex Value for a Fiber

For each of the elastic fibers removed from an absorbent article or specimen extracted from an absorbent article according to the procedure described above, the length of each elastic fiber Lₖ in its relaxed state is measured and recorded in millimeters (mm) to the nearest mm. Each elastic fiber is analyzed via FT-IR spectroscopy to determine its composition, and its density ρₖ is determined from available literature values. Finally, each fiber is analyzed via SEM. The fiber is cut in three approximately equal locations perpendicularly along its length with a sharp blade to create a clean cross-section for SEM analysis. Three fiber segments with these cross sections exposed are mounted on an SEM sample holder in a relaxed state, sputter coated with gold, introduced into an SEM for analysis, and imaged at a resolution sufficient to clearly elucidate fiber cross sections. Fiber cross sections are oriented as perpendicular as possible to the detector to minimize any oblique distortion in the measured cross sections. Fiber cross sections may vary in shape, and some fibers may consist of a plurality of individual filaments. Regardless, the area of each of the three fiber cross sections is determined (for example, using diameters for round fibers, major and minor axes for elliptical fibers, and image analysis for more complicated shapes), and the average of the three areas aₖ for the elastic fiber, in units of micrometers squared (μm²), is recorded to the nearest 0.1 μm². The decitex dₖ of the kth elastic fiber measured is calculated by:

$${d}_{k} = 10 \; 000 m \times {a}_{k} \times {ρ}_{k} \times 1 {0}^{- 6}$$

where dₖ is in units of grams (per calculated 10,000 meter length), aₖ is in units of μm², and ρₖ is in units of grams per cubic centimeter (g/cm³). For any elastic fiber analyzed, the experimentally determined Lₖ and dₖ values are subsequently used in the expression above for Average-Dtex.

### Average-Strand-Spacing

Using a ruler calibrated against a certified NIST ruler and accurate to 0.5 mm, measure the distance between the two distal strands within a section to the nearest 0.5 mm, and then divide by the number of strands in that section−1
Average-Strand-Spacing=d/(n−1) where n>1
report to the nearest 0.1 mm.

### Average-Pre-Strain

The Average-Pre-Strain of a specimen are measured on a constant rate of extension tensile tester (a suitable instrument is the MTS Insight using Testworks 4.0 Software, as available from MTS Systems Corp., Eden Prairie, MN) using a load cell for which the forces measured are within 1% to 90% of the limit of the cell. Articles are conditioned at 23° C.±2 C.° and 50%±2% relative humidity for 2 hours prior to analysis and then tested under the same environmental conditions.

Program the tensile tester to perform an elongation to break after an initial gage length adjustment. First raise the cross head at 10 mm/min up to a force of 0.05N. Set the current gage to the adjusted gage length. Raise the crosshead at a rate of 100 mm/min until the specimen breaks (force drops 20% after maximum peak force). Return the cross head to its original position. Force and extension data is acquired at a rate of 100 Hz throughout the experiment.

Set the nominal gage length to 40 mm using a calibrated caliper block and zero the crosshead. Insert the specimen into the upper grip such that the middle of the test strip is positioned 20 mm below the grip. The specimen may be folded perpendicular to the pull axis, and placed in the grip to achieve this position. After the grip is closed the excess material can be trimmed. Insert the specimen into the lower grips and close. Once again, the strip can be folded, and then trimmed after the grip is closed. Zero the load cell. The specimen should have a minimal slack but less than 0.05 N of force on the load cell. Start the test program.

From the data construct a Force (N) verses Extension (mm). The Average-Pre-Strain is calculated from the bend in the curve corresponding to the extension at which the nonwovens in the elastic are engaged. Plot two lines, corresponding to the region of the curve before the bend (primarily the elastics), and the region after the bend (primarily the nonwovens). Read the extension at which these two lines intersect, and calculate the % Pre-Strain from the extension and the corrected gage length. Record as % Pre-strain 0.1%. Calculate the arithmetic mean of three replicate samples for each elastomeric laminate and Average-Pre-Strain to the nearest 0.1%.

### Whole Article Force Test

The Whole Article Force Test Method is used to measure the Full Stretch Circumference and the Fit Circumference Force of a refastenable pant-style diaper with pre-fastened fastening side features. Specimens of a diaper of interested are mounted on a set of fixture bars on a tensile tester that enable the waist circumference of the diaper to be stretched as if worn, and Full Stretch Circumference and Fit Circumference Force are determined from force versus displacement data. Testing is performed at 23±3° C. and 50±2% relative humidity, and sample articles to be tested are conditioned for at least two hours prior to testing in these same environmental conditions.

### Apparatus

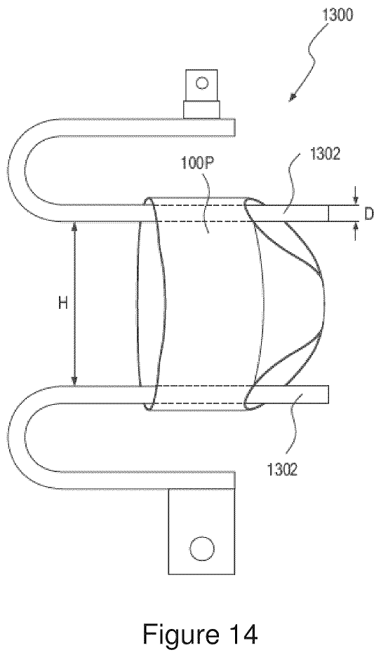

A constant rate of extension tensile tester is equipped with fixture bars 1302 as shown in FIG. 14. Each fixture bar has a round cross section 10.0 mm in diameter and is fashioned from a material that is rigid under the typical for circumferential waist extension of a diaper. The surface of each fixture bar is sufficiently smooth so as not to restrict sliding of the diaper over the surface during stretching. The horizontal bar sections (perpendicular to the axis of crosshead separation of the tensile tester) are sufficient in length to accommodate the entire pre-fastened fastening side features of the diaper and remain parallel and in the same vertical plane throughout the test procedure. At any crosshead position and the gauge circumference is defined by

$$Gauge circumference = 2 H + 2 D + π D$$

where H and D are the separation between horizontal bars and the bar diameter, respectively, as shown in FIG. 14.

### Procedure

The specimen diaper is placed onto the upper horizontal bar such that the bar passes through the waist opening and one leg opening of the diaper specimen and such that the pre-fastened refastenable side feature remains fully closed. The crosshead is raised until the diaper specimen hangs above the lower bar but does not touch the lower bar. The load cell is then tared, and the crosshead is lowered to enable the easy insertion of the lower bar through the waist opening and other leg opening without stretching the diaper specimen. The diaper specimen is adjusted at this point such that the longitudinal centerline of both the front and rear of the diaper specimen is located halfway between the upper and lower bars. The longitudinal center of the side portions in contact with the bars are situated on the axis between the upper and lower crosshead attachment points and load cell.

The crosshead is then raised slowly while the diaper specimen is stabilized by hand (as necessary to maintain the required position) until the force is 0.075±0.025 N. The crosshead then is moved upward (separating the bars) at 254.0 mm/min until a final load of 19.61 N is reached, at which point the crosshead immediately is returned to its initial position at 254.0 mm/min. Data are acquired throughout at 50 Hz. The gauge circumference at maximum load (19.61 N) is determined and recorded to the nearest 0.1 mm as the full stretch circumference for that diaper specimen. The load corresponding to 75% of the full stretch circumference on the latter, returning half of the tensile tester movement is determined and recorded as the fit circumference force to the nearest 0.01 N for that diaper specimen.

Five like diaper specimens of a diaper of interest are analyzed as specified. The arithmetic mean of the full stretch circumferences of among the five diaper specimens is calculated and reported to the nearest 0.1 mm as the Full Stretch Circumference of the diaper. The arithmetic mean of the fit circumference force of among the five diaper specimens is calculated and reported to the nearest 0.01 N as the Fit Circumference Force of the diaper.

### Belt Zone Tensile Force Method

The Belt Zone Tensile Force Test Method is used to characterize the tensile properties of one belt zone of interest (e.g., belt zones 502, 504, 506, 522, 524, or 526) from a refastenable pant-style diaper of interest. This method makes use of the Full Stretch Circumference as measured by the Whole Article Force Test Method. A belt zone of interest is excised from a finished article and is extended on a constant rate of extension tensile tester. The force at point of interest on the crosshead extension is recorded. Testing is performed at 23±3° C. and 50±2% relative humidity, and sample articles to be tested are conditioned for at least two hours prior to testing in these same environmental conditions.

### Sample Preparation

Prior to the excision of any belt zone of interest and prior to separating pre-fastened refastenable side feature, the distance between longitudinally running inboard edges and outboard edges of each refastenable side feature is marked on both the front and rear belt sections. The side features are then separated, and the distance between the inboard and outboard edges of each side feature are measured and recorded. For any article, the arithmetic mean of these two measurements is recorded as the fastener width for that article.

On five like articles, a belt zone of interest is then excised. This may generally involve first separating the front and rear belt sections from the main body before sectioning the belt zone of interest. Cryogenic freeze spray may be used but is used to as to minimally affect the bonding of elastics within the belt elastic body.

### Apparatus and Procedure

A constant rate of extension tensile tester is configured with grips with width at least as great as the longitudinal extent of the belt zone to measured. The tensile tester is set to have an initial gauge length of 90 mm. The final gauge length makes use of the result from the Whole Article Force Test Method. In particular, final gauge length is set to be the difference between half of the Full Stretch Circumference and the fastener width (measured for the particular article as described above).

A belt zone specimen is mounted in the tensile tester so that the lateral extent of the belt zone is oriented along the crosshead movement axis of the tensile tester (such that the tensile pull is somewhat akin to the circumferential pull on the full article). The clamps are positioned on the belt zones so that the edge of the clamp defining the gauge are coincident with the markings denoting the laterally inboard edges of the side features. The clamps are then separated at 254.0 mm/min until the final gauge length is reached, at which point the clamps immediately are returned at a rate 254.0 mm/min toward and terminating at their original separation of 90 mm. Force data are acquired at 50 Hz throughout, and force versus absolute displacement data are recorded. All five like belt zone specimens are analyzed in this way.

### Analysis and Reporting

For each of the five like belt zone test specimens, the force at 75% of the final gauge length on the extension segment of the test pull is recorded. The arithmetic mean of these five force values is calculated and reported in Newtons (N) to the nearest 0.01 N as the belt zone tensile force for the belt zone of interest (e.g., belt zone 502, 504, 506, 522, 524, or 526) being tested.

### Combinations

A1. An absorbent article comprising: a chassis comprising a topsheet, a backsheet, and an absorbent core positioned between the topsheet and the backsheet, the chassis further comprising a first end region and a second end region longitudinally separated from the first end region by a crotch region; a first belt comprising a first end region and a second end region laterally separated from the first end region by a central region, the first end region of the chassis connected with the central region of the first belt; a second belt comprising a first end region and a second end region laterally separated from the first end region by a central region, the second end region of the chassis connected with the central region of the second belt; wherein the first end region and the second end region of the first belt each comprise a fastener component; wherein the fastener components are refastenably connected with the first and second end regions of the second belt to form a waist opening and two leg openings; wherein the first and second belts each comprise: a wearer facing surface and a garment facing surface; an outer laterally extending edge extending along a portion of the waist opening; an inner laterally extending edge extending along a portion of each of the leg openings; and a first substrate, a second substrate, and a plurality of elastic strands positioned between and connected with the first substrate and the second substrate; wherein a portion of the chassis overlaps the wearer facing surface of the first belt to define a chassis overlap region, wherein the chassis overlap region comprises an attachment area where adhesive is positioned between the chassis and the wearer facing surface of the first belt and bonds the chassis with the first belt; wherein the attachment area comprises a first attachment zone adjacent to the inner laterally extending edge of the first belt and a second attachment zone positioned longitudinally outboard of the first attachment zone, wherein the first attachment zone comprises a first lateral width and the second attachment zone comprises a second lateral width, wherein the first lateral width is less than the second lateral width; wherein the first belt comprises a first lower elastic zone extending in a lateral direction and positioned longitudinally inboard of the second attachment zone and laterally outboard of the first attachment zone and a first upper elastic zone extending in the lateral direction and positioned longitudinally outboard of the first lower elastic zone, wherein the first lower elastic zone has a tensile force of from 0.10 N to 1.20 N.
A2. The absorbent article of paragraph A1, wherein the first lateral width is from 10% to 15% less than the second lateral width.
A3. The absorbent article of any A1 or A2, wherein the first lateral width is from 75% to 85% of a lateral width of the chassis overlap region.
A4. The absorbent article of any of paragraphs A1 to A3, wherein the first attachment zone comprises a first longitudinal length and the second attachment zone comprises a second longitudinal length, wherein the first longitudinal length is less than the second longitudinal length.
A5. The absorbent article of any of paragraphs A1 to A4, wherein the second attachment zone comprises laterally extending strips of adhesive longitudinally separated from each other.
A6. The absorbent article of any of paragraphs A1 to A5, wherein the chassis overlap region further comprises a first non-bonded region and a second non-bonded region, wherein the first non-bonded region is positioned longitudinally inboard of the second attachment zone and extends laterally between first attachment zone and a first side edge of the chassis, wherein the second non-bonded region is positioned longitudinally inboard of the second attachment zone and extends laterally between the first attachment zone and a second side edge of the chassis, wherein no adhesive connects the chassis with the first belt in the overlap region in the first non-bonded region and the second non-bonded region.
A7. The absorbent article of any of paragraphs A1 to A6, wherein the absorbent article comprises a first flange comprising a first surface and a second surface opposite the first surface, the first flange further comprising a first end region and a second end region, wherein the first surface of the first end region of the first flange is bonded with the wearer facing surface of the first end region of the first belt, wherein the second surface of the second end region of the first flange is in a facing relationship with the wearer facing surface of the second belt, wherein at least one fastener component is positioned on the second surface of the first flange.
A8. The absorbent article of paragraph A7, wherein the absorbent article comprises a second flange comprising a first surface and a second surface opposite the first surface, the second flange further comprising a first end region and a second end region, wherein the first surface of the first end region of the second flange is bonded with the wearer facing surface of the second end region of the first belt, wherein the second surface of the second end region of the second flange is in a facing relationship with the wearer facing surface of the second belt, wherein at least one fastener component is positioned on the second surface of the second flange.
A9. The absorbent article of any of paragraphs A1 to A8, wherein the fastener components comprise a plurality of protrusions extending from a base.
A10. The absorbent article of paragraph A7, wherein the fastener component comprise a plurality of protrusions integrally formed from a material of the first flange.
A11. The absorbent article of any of paragraphs A1 to A10, wherein the fastener components comprise a first end edge and a longitudinally opposed second end edge, wherein the second end edge of the fastener components are positioned longitudinally inboard of the second attachment zone and laterally outboard of the first attachment zone.
A12. The absorbent article of paragraph All, wherein the first lower elastic zone is adjacent to the second end edge of the fastener components.
A13. The absorbent article of any of paragraphs A1 to A12, wherein the fastener components comprise a first end edge positioned adjacent to the outer laterally extending edge of the first belt and a longitudinally opposed second end edge, wherein the plurality of elastic strands comprise at least a first elastic strand positioned adjacent to the outer laterally extending edge of the first belt, and wherein the first elastic strand and the first end edge of the fastener components are spaced apart from each other by a distance of from 0 to 8 mm.
A14. The absorbent article of any of paragraphs A1 to A13, wherein the fastener components comprise a first end edge positioned adjacent to the outer laterally extending edge of the first belt and a longitudinally opposed second end edge, wherein the plurality of elastic strands comprise at least a first elastic strand and a second elastic strand, wherein the first end edge of the fastener component is positioned between the first elastic strand and the second elastic strand.
A15. The absorbent article of any of paragraphs A1 to A14, further comprising a front waist region and a back waist region, wherein the first belt is positioned in the front waist region and the second belt is positioned in the back waist region.
A16. The absorbent article of paragraph A7, wherein the first flange comprises a first end edge and a longitudinally opposed second end edge, wherein the fastener component comprises a first end edge and a longitudinally opposed second end edge, wherein the first end edge of the fastener component is positioned longitudinally inboard of the first end edge of the first flange by a distance of from 2 mm to 8 mm, preferably from 4 mm to 6 mm.
A17. The absorbent article paragraph A16, wherein the second end edge of the fastener component is positioned longitudinally inboard of the second end edge of the first flange by a distance of from 2 mm to 8 mm, preferably from 4 mm to 6 mm.
A18. The absorbent article of paragraph A7, wherein the first end region of the first flange comprises a first side edge, wherein the fastener component comprises a first side edge and a laterally opposed second side edge, wherein the first side edge of the fastener component is positioned laterally in board of the first side edge of the first flange a distance of from 8 mm to 21 mm, preferably from 10 mm to 18 mm.
A19. The absorbent article of paragraph A7, wherein the second end region of the first flange comprises a second side edge, wherein the fastener component comprises a first side edge and a laterally opposed second side edge, wherein the second side edge of the fastener component is positioned laterally in board of the second side edge of the first flange a distance of from 1 mm to 9 mm, preferably from 3 mm to 7 mm.
A20. The absorbent article of claim 7, wherein the first flange has a lateral width of from 26 mm to 40 mm, preferably from 30 mm to 38 mm.

### Bio-Based Content for Components

Components of the absorbent articles described herein may at least partially be comprised of bio-based content as described in U.S. Pat. Appl. No. 2007/0219521 A1. For example, the superabsorbent polymer component may be bio-based via their derivation from bio-based acrylic acid. Bio-based acrylic acid and methods of production are further described in U.S. Pat. Appl. Pub. No. 2007/0219521 and U.S. Pat. Nos. 8,703,450; 9,630,901 and 9,822,197. Other components, for example nonwoven and film components, may comprise bio-based polyolefin materials. Bio-based polyolefins are further discussed in U.S. Pat. Appl. Pub. Nos. 2011/0139657, 2011/0139658, 2011/0152812, and 2016/0206774, and U.S. Pat. No. 9,169,366. Example bio-based polyolefins for use in the present disclosure comprise polymers available under the designations SHA7260™, SHE150™, or SGM9450F™ (all available from Braskem S.A.).

An absorbent article component may comprise a bio-based content value from about 10% to about 100%, from about 25% to about 100%, from about 40% to about 100%, from about 50% to about 100%, from about 75% to about 100%, or from about 90% to about 100%, for example, using ASTM D6866-10, method B.

### Recycle Friendly and Bio-Based Absorbent Articles

Components of the absorbent articles described herein may be recycled for other uses, whether they are formed, at least in part, from recyclable materials. Examples of absorbent article materials that may be recycled are nonwovens, films, fluff pulp, and superabsorbent polymers. The recycling process may use an autoclave for sterilizing the absorbent articles, after which the absorbent articles may be shredded and separated into different byproduct streams. Example byproduct streams may comprise plastic, superabsorbent polymer, and cellulose fiber, such as pulp. These byproduct streams may be used in the production of fertilizers, plastic articles of manufacture, paper products, viscose, construction materials, absorbent pads for pets or on hospital beds, and/or for other uses. Further details regarding absorbent articles that aid in recycling, designs of recycle friendly diapers, and designs of recycle friendly and bio-based component diapers, are disclosed in U.S. Pat. Appl. Publ. No. 2019/0192723, published on Jun. 27, 2019.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.” Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An absorbent article comprising:
a chassis comprising a topsheet, a backsheet, and an absorbent core positioned between the topsheet and the backsheet, the chassis further comprising a first end region and a second end region longitudinally separated from the first end region by a crotch region;
a first belt comprising a first end region and a second end region laterally separated from the first end region by a central region, the first end region of the chassis connected with the central region of the first belt;
a second belt comprising a first end region and a second end region laterally separated from the first end region by a central region, the second end region of the chassis connected with the central region of the second belt;
wherein the first end region and the second end region of the first belt each comprise a fastener component;
wherein the fastener components are refastenably connected with the first and second end regions of the second belt to form a waist opening and two leg openings;
wherein the first and second belts each comprise:
a wearer facing surface and a garment facing surface;
an outer laterally extending edge extending along a portion of the waist opening;
an inner laterally extending edge extending along a portion of each of the leg openings; and
a first substrate, a second substrate, and a plurality of elastic strands positioned between and connected with the first substrate and the second substrate;
wherein a portion of the chassis overlaps the wearer facing surface of the first belt to define a chassis overlap region, wherein the chassis overlap region comprises an attachment area where adhesive is positioned between the chassis and the wearer facing surface of the first belt and bonds the chassis with the first belt;
wherein the attachment area comprises a first attachment zone adjacent to the inner laterally extending edge of the first belt and a second attachment zone positioned longitudinally outboard of the first attachment zone, wherein the first attachment zone comprises a first lateral width and the second attachment zone comprises a second lateral width, wherein the first lateral width is from 10% to 15% less than the second lateral width.

2. The absorbent article of claim 1, wherein the chassis overlap region further comprises a first non-bonded region and a second non-bonded region, wherein the first non-bonded region is positioned longitudinally inboard of the second attachment zone and extends laterally between first attachment zone and a first side edge of the chassis, wherein the second non-bonded region is positioned longitudinally inboard of the second attachment zone and extends laterally between the first attachment zone and a second side edge of the chassis, wherein no adhesive connects the chassis with the first belt in the overlap region in the first non-bonded region and the second non-bonded region.

3. The absorbent article of claim 1, wherein the first lateral width is from 75% to 85% of a lateral width of the chassis overlap region.

4. The absorbent article of claim 1, wherein the absorbent article comprises a flange connected with opposing end regions of the first belt; wherein the flange comprises at least one fastener component.

5. The absorbent article of claim 1, wherein the fastener components comprise a plurality of protrusions extending from a base.

6. An absorbent article comprising:
a chassis comprising a topsheet, a backsheet, and an absorbent core positioned between the topsheet and the backsheet, the chassis further comprising a first end region and a second end region longitudinally separated from the first end region by a crotch region;
a first belt comprising a first end region and a second end region laterally separated from the first end region by a central region, the first end region of the chassis connected with the central region of the first belt;
a second belt comprising a first end region and a second end region laterally separated from the first end region by a central region, the second end region of the chassis connected with the central region of the second belt;
wherein the first end region and the second end region of the first belt each comprise a fastener component;
wherein the fastener components are refastenably connected with the first and second end regions of the second belt to form a waist opening and two leg openings;
wherein the first and second belts each comprise:
a wearer facing surface and a garment facing surface;
an outer laterally extending edge extending along a portion of the waist opening;
an inner laterally extending edge extending along a portion of each of the leg openings; and
a first substrate, a second substrate, and a plurality of elastic strands positioned between and connected with the first substrate and the second substrate;
wherein a portion of the chassis overlaps the wearer facing surface of the first belt to define a chassis overlap region, wherein the chassis overlap region comprises an attachment area where adhesive is positioned between the chassis and the wearer facing surface of the first belt and bonds the chassis with the first belt;
wherein the attachment area comprises a first attachment zone adjacent to the inner laterally extending edge of the first belt and a second attachment zone positioned longitudinally outboard of the first attachment zone, wherein the first attachment zone comprises a first lateral width and the second attachment zone comprises a second lateral width, wherein the first lateral width is less than the second lateral width;
wherein the first belt comprises a first lower elastic zone extending in a lateral direction and positioned longitudinally inboard of the second attachment zone and laterally outboard of the first attachment zone and a first upper elastic zone extending in the lateral direction and positioned longitudinally outboard of the first lower elastic zone, wherein the first lower elastic zone has a tensile force of less than 1.2 N.

7. The absorbent article of claim 6, wherein the first lateral width is from 10% to 15% less than the second lateral width.

8. The absorbent article of claim 6, wherein the tensile force of the first lower elastic zone is from 0.1 N to 1.2N.

9. The absorbent article of claim 6, wherein the first lateral width is from 75% to 85% of a lateral width of the chassis overlap region.

10. The absorbent article of claim 6, wherein the first attachment zone comprises a first longitudinal length and the second attachment zone comprises a second longitudinal length, wherein the first longitudinal length is less than the second longitudinal length.

11. The absorbent article of claim 6, wherein the second attachment zone comprises laterally extending strips of adhesive longitudinally separated from each other.

12. The absorbent article of claim 6, wherein the chassis overlap region further comprises a first non-bonded region and a second non-bonded region, wherein the first non-bonded region is positioned longitudinally inboard of the second attachment zone and extends laterally between first attachment zone and a first side edge of the chassis, wherein the second non-bonded region is positioned longitudinally inboard of the second attachment zone and extends laterally between the first attachment zone and a second side edge of the chassis, wherein no adhesive connects the chassis with the first belt in the overlap region in the first non-bonded region and the second non-bonded region.

13. The absorbent article of claim 6, wherein the absorbent article comprises a first flange comprising a first surface and a second surface opposite the first surface, the first flange further comprising a first end region and a second end region, wherein the first surface of the first end region of the first flange is bonded with the wearer facing surface of the first end region of the first belt, wherein the second surface of the second end region of the first flange is in a facing relationship with the wearer facing surface of the second belt, wherein at least one fastener component is positioned on the second surface of the first flange.

14. The absorbent article of claim 13, wherein the first flange comprises a first end edge and a longitudinally opposed second end edge, wherein the fastener component comprises a first end edge and a longitudinally opposed second end edge, wherein the first end edge of the fastener component is positioned longitudinally inboard of the first end edge of the first flange by a distance of from about 2 mm to about 8 mm.

15. The absorbent article of claim 13, wherein the first end region of the first flange comprises a first side edge, wherein the fastener component comprises a first side edge and a laterally opposed second side edge, wherein the first side edge of the fastener component is positioned laterally in board of the first side edge of the first flange a distance of from about 8 mm to about 21 mm.

16. The absorbent article of claim 6, wherein the fastener components comprise a first end edge and a longitudinally opposed second end edge, wherein the second end edge of the fastener components are positioned longitudinally inboard of the second attachment zone and laterally outboard of the first attachment zone.

17. The absorbent article of claim 16, wherein the first lower elastic zone is adjacent to the second end edge of the fastener components.

18. The absorbent article of claim 6, wherein the fastener components comprise a first end edge positioned adjacent to the outer laterally extending edge of the first belt and a longitudinally opposed second end edge, wherein the plurality of elastic strands comprise at least a first elastic strand positioned adjacent to the outer laterally extending edge of the first belt, and wherein the first elastic strand and the first end edge of the fastener components are spaced apart from each other by a distance of from 0 to 8 mm.

19. The absorbent article of claim 6, wherein the plurality of elastic strands comprise at least a first elastic strand and a second elastic strand, wherein the first end edge of the fastener component is positioned between the first elastic strand and the second elastic strand.

20. The absorbent article of claim 6, further comprising a front waist region and a back waist region, wherein the first belt is positioned in the front waist region and the second belt is positioned in the back waist region.
